(19) United States
(12) Patent Application Publication
(10) Pub. No.: US 2007/0054281 A1
(43) Pub. Date: Mar. 8, 2007

(54) Compositions and methods relating to osteoarthritis

(75) Inventors: Choong-Chin Liew, Toronto (CA); Hongwei Zhang, Toronto (CA); K. Wayne Marshall, Toronto (CA)

Correspondence Address: PALMER & DODGE, LLP;KATHLEEN M. WILLIAMS, 111 HUNTINGTON AVENUE, BOSTON, MA, 02199, US

(73) Assignee: Chrondrogene Inc.

(21) Appl. No.: 11/272,125

(22) Filed: Nov. 10, 2005

Related U.S. Application Data
(60) Provisional application No. 60/626,511, filed on Nov. 10, 2004.

(51) Int. Cl. C12Q 1/68

(52) U.S. Cl. 435/6

(57) ABSTRACT

Described are compositions and methods related to the identification, selection and use of biomarkers which demonstrate particular advantage in identifying individuals having osteoarthritis. Also described are compositions and methods related to the identification, selection and use of biomarkers which demonstrate particular advantage in identifying individuals having a particular stage of Osteoarthritis. Also presented are methods for quantitating the biomarkers in a sample from an individual, for use in the diagnosis and/or prognosis of osteoarthritis.

### RELATED APPLICATIONS

This application claims the benefit of U.S. Provisional Application No. 60/626,511 filed on Nov. 10, 2004, which is incorporated herein by reference in its entirety, including figures and drawings.

### TABLES

This application includes a compact disc in duplicate (2 compact discs: Tables—Copy 1 and Tables—Copy 2), which are hereby incorporated by reference in their entirety. Each compact disc is identical and contains the following files (corresponding to Tables 1-7):

| TABLE | DESCRIPTION | SIZE | | CREATED | Text File Name |
|---|---|---|---|---|---|
| 1 | shows all genes identified as | 1,424 | KB | 11/6/2004 | RevTable1.TXT |
| | from one or more of the | | | | |
| | cDNA libraries derived from | | | | |
| | chondrocytes categorized as | | | | |
| | follows: normal, mild OA, and | | | | |
| | severe OA. Table 1 indicates | | | | |
| | the frequency of the EST as | | | | |
| | isolated from each library. | | | | |
| | Also indicated is a call as to | | | | |
| | whether a gene is up | | | | |
| | regulated, down regulated or | | | | |
| | shows no regulation as | | | | |
| | compared with the normal | | | | |
| | library as determined based on | | | | |
| | EST frequency. A p-value | | | | |
| | indicative of the statistical | | | | |
| | likelihood of the call being | | | | |
| | determined by chance is also | | | | |
| | provided. | | | | |
| 2 | Genes identified as mild | 312 | KB | 11/3/2004 | RevTable2.TXT |
| | Osteoarthritis biomarkers on | | | | |
| | the basis of the query outlined | | | | |
| | in the decision matrix of | | | | |
| | FIG. 1. | | | | |
| 3 | Genes identified as severe | 295 | KB | 10/29/2004 | RevTAB3.TXT |
| | Osteoarthritis biomarkers on | | | | |
| | the basis of the query outlined | | | | |
| | in the decision matrix of | | | | |
| | FIG. 2. | | | | |
| 4 | Genes identified as mild | 11 | KB | 10/28/2004 | RevTAB4.TXT |
| | Osteoarthritis biomarkers on | | | | |
| | the basis of the query outlined | | | | |
| | in the decision matrix of | | | | |
| | FIG. 3. | | | | |
| 5 | Genes identified as severe | 14 | KB | 10/28/2004 | RevTAB5.TXT |
| | Osteoarthritis biomarkers on | | | | |
| | the basis of the query outlined | | | | |
| | in the decision matrix of | | | | |
| | FIG. 4. | | | | |
| 6 | Genes identified as mild | 3 | KB | 10/28/2004 | RevTAB6.TXT |
| | Osteoarthritis biomarkers on | | | | |
| | the basis of the query outlined | | | | |
| | in the decision matrix of | | | | |
| | FIG. 5. | | | | |
| 7 | Genes identified as severe | 4 | KB | 10/28/2004 | RevTable 7.TXT |
| | Osteoarthritis biomarkers on | | | | |
| | the basis of the query outlined | | | | |
| | in the decision matrix of | | | | |
| | FIG. 6. | | | | |

LENGTHY TABLES FILED ON CD
The patent application contains a lengthy table section. A copy of the table is available in electronic form from the USPTO web site (http://seqdata.uspto.gov/?pageRequest=docDetail&DocID=US20070054281A1). An electronic copy of the table will also be available from the USPTO upon request and payment of the fee set forth in 37 CFR 1.19(b)(3).

### FIELD OF THE INVENTION

The invention relates to the profiling of differential gene expression in human tissue cartilage through the construction and use of cDNA libraries and microarrays. The invention identifies more that 5,000 genes which are transcribed in human cartilage and differentially regulated in chondrocytes from individuals with severe or mild Osteoarthritis, and in chondrocytes from individuals without osteoarthritis. Using EST frequency analysis, the invention further qualifies genes as biomarkers on the basis of being up regulated or downregulated in chondrocytes from individuals with severe or mild Osteoarthritis, as compared with the same genes in chondrocytes from individuals without osteoarthritis. The invention also identifies stage specific biomarkers which are useful in differentiating mild Osteoarthritis from severe Osteoarthritis and in particular presents 28 biomarkers which are particularly useful for the diagnosis and prognosis of mild Osteoarthritis. Also disclosed are 33 biomarkers which are particularly useful for the diagnosis and prognosis of severe Osteoarthritis. The invention also presents methods for quantitating the biomarkers in a sample from an individual, for use in the diagnosis and/or prognosis of osteoarthritis.

### BACKGROUND OF THE INVENTION

Osteoarthritis (OA) is a chronic disease in which the articular cartilage that lies on the ends of bones that forms the articulating surface of the joints gradually degenerates over time. There are many factors that are believed to predispose a patient to Osteoarthritis including genetic susceptibility, obesity, accidental or athletic trauma, surgery, drugs and heavy physical demands. Osteoarthritis is initiated by damage to the cartilage of joints. The two most common injuries to joints are sports-related injuries and long term “repetitive use” joint injuries. Joints most commonly affected by Osteoarthritis are the knees, hips and hands. In most cases, due to the essential weight-bearing function of the knees and hips, Osteoarthritis in these joints causes much more disability than Osteoarthritis of the hands. As cartilage degeneration progresses, secondary changes occur in other tissues in and around joints including bone, muscle, ligaments, menisci and synovium. The net effect of the primary failure of cartilage tissue and secondary damage to other tissues is that the patient experiences pain, swelling, weakness and loss of functional ability in the afflicted joint(s). These symptoms frequently progress to the point that they have a significant impact in terms of lost productivity and or quality of life consequences for the patient.

Articular cartilage is predominantly composed of chondrocytes, type II collagen, proteoglycans and water. Articular cartilage has no blood or nerve supply and chondrocytes are the only type of cell in this tissue. Chondrocytes are responsible for manufacturing the type II collagen and proteoglycans that form the cartilage matrix. This matrix in turn has physical-chemical properties that allow for saturation of the matrix with water. The net effect of this structural-functional relationship is that articular cartilage has exceptional wear characteristics and allows for almost frictionless movement between the articulating cartilage surfaces. In the absence of Osteoarthritis, articular cartilage often provides a lifetime of pain-free weight bearing and unrestricted joint motion even under demanding physical conditions.

During fetal development, articular cartilage is initially derived from the interzone of mesenchymal condensations. The mesenchymal cells cluster together and synthesize matrix proteins. The tissue is recognized as cartilage when the accumulation of matrix separates the cells, which are spherical in shape and are now called chondrocytes. During cartilage formation and growth, chondrocytes proliferate rapidly and synthesize large volumes of matrix. Prior to skeletal maturity, chondrocytes are at their highest level of metabolic activity. As skeletal maturation is reached, the rate of chondrocyte metabolic activity and cell division declines. After completion of skeletal growth, most chondrocytes do not divide but do continue to synthesize matrix proteins such as collagens, proteoglycans and other noncollagenous proteins (1, 2).

Like all living tissues, articular cartilage is continually undergoing a process of renewal in which “old” cells and matrix components are being removed (catabolic activity) and “new” cells and molecules are being produced (anabolic activity). Relative to most tissues, the rate of anabolic/catabolic turnover in articular cartilage is low. Long-term maintenance of the structural integrity of mature cartilage relies on the proper balance between matrix synthesis and degradation. Chondrocytes maintain matrix equilibrium by responding to chemical and mechanical stimuli from their environment. Appropriate and effective chondrocyte responses to these stimuli are essential for cartilage homeostasis. Disruption of homeostasis through either inadequate anabolic activity or excessive catabolic activity can result in cartilage degradation and Osteoarthritis (3). Most tissues that are damaged and have increased catabolic activity are able to mount an increased anabolic response that allows for tissue healing. Unfortunately, chondrocytes have very limited ability to up-regulate their anabolic activity and increase the synthesis of proteoglycan and type II collagen in response to damage or loss of cartilage matrix. This fundamental limitation of chondrocytes is the core problem that has precluded the development of therapies that can prevent and cure Osteoarthritis. Additionally, there is a need for a definitive diagnostic test for detecting early (mild) Osteoarthritis, and a prognostic test that effectively monitors a patient's response to therapy, for example in the treatment of severe or mild Osteoarthritis.

Joint pain is the most common manifestation of early Osteoarthritis. The pain tends to be episodic lasting days to weeks and remitting spontaneously. Although redness and swelling of joints is uncommon, joints become tender during a flare-up of Osteoarthritis.

“Mild” or “early stage Osteoarthritis” is difficult to diagnose. The physician relies primarily on the patient's history and physical exam to make the diagnosis of mild Osteoarthritis. X-rays do not show the underlying early changes in articular cartilage. There are no recognized biochemical markers used to confirm the diagnosis of early stage Osteoarthritis.

X-ray changes confirm the diagnosis of moderate Osteoarthritis. X-rays of normal joints reveal well preserved symmetrical joint spaces. Changes seen on the x-rays of patients with Osteoarthritis include new bone formation (osteophytes), joint space narrowing and sclerosis (bone thickening). There are no recognized biochemical markers used to confirm the diagnosis of “moderate Osteoarthritis” at this stage.

The clinical exam of a joint with severe Osteoarthritis reveals tenderness, joint deformity and a loss of mobility. Passive joint movement during examination may elicit crepitus or the grinding of bone-on-bone as the joint moves. X-ray changes are often profound: the joint space may be obliterated and misalignment of the joint can be seen. New bone formation (osteophytes) is prominent. Again, there are no recognized biochemical markers used to confirm the diagnosis of “severe Osteoarthritis”.

“Osteoarthritis” is the most common chronic joint disease. It is characterized by progressive degeneration and eventual loss of cartilage. Currently, there is a need for an effective therapy that will alter the course of Osteoarthritis. Further advances in preventing, modifying or curing the osteoarthritic disease process critically depends, at least in part, on a thorough understanding of the molecular mechanisms underlying anabolic and catabolic processes in cartilage. Since cellular functions are substantially determined by the genes that the cells express, elucidating the genes expressed in articular cartilage at different developmental and disease stages will inevitably provide new insights into the molecules and mechanisms involved in cartilage formation, injury, disease and repair.

cDNA libraries from putatively normal and severe osteoarthritic human cartilage tissue have been constructed (Kumar et al., 46^{th} Annual Meeting, Orthopaedic Res. Soc., Abstract, p. 1031). However, this work does not adequately address the differential gene expression in chondrocytes from differing severities of osteoarthritic human cartilage (i.e. between mild and severe). In addition, the “normal cartilage” samples were obtained from deceased donors more than 24 hours after death. Thus, this cDNA library does not truly reflect normal chondrocyte gene expression due to the rapid degeneration of RNA that occurs after cessation of perfusion to the sampled joint, as demonstrated by baboon studies, presented herein below.

More importantly previous studies have not identified sequences which will be effective in diagnosing Osteoarthritis or in diagnosing the degree of advancement of Osteoarthritis so as to aid in both early detection, and treatment.

### SUMMARY OF THE INVENTION

The present invention identifies more than 5,000 genes which are transcribed in human cartilage and differentially regulated in diseased chondrocytes. Using EST frequency analysis, the invention qualifies genes as biomarkers on the basis of being up regulated or downregulated in chondrocytes from individuals with severe or mild Osteoarthritis, as compared with the same genes in chondrocytes from individuals without osteoarthritis. The invention also identifies stage specific biomarkers which are useful in differentiating mild Osteoarthritis from severe Osteoarthritis and in particular presents 28 biomarkers which are particularly useful for the diagnosis and prognosis of mild Osteoarthritis. Also disclosed are 33 biomarkers which are particularly useful for the diagnosis and prognosis of severe Osteoarthritis. The invention also presents methods for quantitating the biomarkers in a sample from an individual, for use in the diagnosis and/or prognosis of osteoarthritis.

To elucidate the transcriptional events underlying cartilage dysfunction (e.g. osteoarthritis) we characterized the complete set of genes expressed in human cartilage. This led to our large-scale generation and sequencing of ESTs derived from cartilage samples obtained from normal adults, and adults with osteoarthritis. Specifically, we have examined a set of cDNA libraries derived from true biological samples (cartilage from normal adults, cartilage from adults with mild OA, and cartilage from adults with severe OA). Table 1 displays the 5,687 genes not previously associated with cartilage dysfunction. These genes are differentially expressed as between the osteoarthritic libraries and the normal libraries and therefore are useful as biomarkers of osteoarthritis. In addition, some of these genes are additionally identified as differentiating between mild osteoarthritis and other stages of osteoarthritis, or as between severe osteoarthritis and other stages of osteoarthritis and are therefore useful as stage specific biomarkers.

As a means of measuring differential gene expression in the cartilage libraries, the EST frequency for each of these 5,687 genes in each of the libraries was determined using over 110,000 ESTs. Based on the relative EST frequencies, each of the 5,687 genes was classified as differentially expressed when comparing mild osteoarthritic cartilage and cartilage from normal adults and when comparing severe osteoarthritic cartilage and cartilage from normal adults. For each of these comparisons, the genes were identified as either “up regulated”, “down regulated” or “no regulation as between diseased cartilage and normal cartilage (e.g. for both mild and severe osteoarthritic cartilage) (see Table 1). A p-value ranking of each call of “up regulated” “down regulated” or “no regulation” is also provided in accordance with the method outlined by Stephane Audic and J-M Clayerie. The results of these analyses are also shown in Table 1.

Identification of stage specific biomarkers useful in differentiating as between stages of OA and in particular, differentiating between mild osteoarthritis and severe osteoarthritis was determined using the call of “up regulated” “down regulated” or “no regulation” in accordance with the decision matrices shown in FIGS. 1-6. FIGS. 1, 3, and 5 provides decision matrices used to identify subsets of mild osteoarthritis specific biomarkers. FIGS. 2, 4, and 6 provide decision matrices used to identify subsets of severe osteoarthritis specific biomarkers.

Products of biomarkers can be measured using numerous techniques so as to allow prognostic or diagnostic determinations of status with respect to osteoarthritis. For example, using quantitative RT-PCR or hybridization techniques to measure the level of the mRNA of one or more biomarkers as disclosed herein will allow the ability to diagnose an individual as having OA, or as having a particular stage of OA. The ability to monitor the RNA products of the biomarkers can also be used to assess the efficacy of a particular therapeutic treatment.

For microarray analysis, the level of expression is measured by hybridization analysis using labeled target nucleic acids according to methods well known in the art. The label on the target nucleic acid can be a luminescent label, an enzymatic label, a radioactive label, a chemical label or a physical label. Preferably, target nucleic acids are labeled with a fluorescent molecule. Preferred fluorescent labels include, but are not limited to: fluorescein, amino coumarin acetic acid, tetramethylrhodamine isothiocyanate (TRITC), Texas Red, Cyanine 3 (Cy3) and Cyanine 5 (Cy5). RT-PCR analysis is another means of identifying or confirming OA biomarkers, and can be used in methods that diagnose or prognose OA based on the differential expression of OA biomarkers.

Also encompassed by the invention are in vitro and in vivo methods of screening for candidate therapeutic molecules.

### BRIEF DESCRIPTION OF THE DRAWINGS

The objects and features of the invention can be better understood with reference to the following detailed description and drawings.

FIG. 1—illustrates, in one embodiment of the invention, a decision matrix used to identify a group of mild specific Osteoarthritis biomarkers.

FIG. 2—illustrates, in one embodiment of the invention, a decision matrix used to identify a group of severe specific Osteoarthritis biomarkers.

FIG. 3—illustrates, in one embodiment of the invention, a decision matrix used to identify a smaller subset of mild specific Osteoarthritis biomarkers than identified using decision matrix identified in FIG. 1.

FIG. 4—illustrates, in one embodiment of the invention, a decision matrix used to identify a smaller subset of severe specific Osteoarthritis biomarkers than identified using decision matrix identified in FIG. 2.

FIG. 5—illustrates, in one embodiment of the invention, a decision matrix used to identify a smaller subset of mild specific Osteoarthritis biomarkers than identified using either the decision matrix identified in FIG. 1 or FIG. 3.

FIG. 6—illustrates, in one embodiment of the invention, a decision matrix used to identify a smaller subset of severe specific Osteoarthritis biomarkers than identified using either the decision matrix identified in FIG. 2 or FIG. 4.

Table 1—shows all genes identified as from one or more of the cDNA libraries derived from chondrocytes categorized as follows: normal, mild OA, and severe OA. Table 1 indicates the frequency of the EST as isolated from each library. Also indicated is a call as to whether a gene is up regulated, downregulated or shows no regulation as compared with the normal library as determined based on EST frequency. A p-value indicative of the statistical likelihood of the call being determined by chance is also provided.

Table 2—Genes identified as mild Osteoarthritis biomarkers on the basis of the query outlined in the decision matrix of FIG. 1.

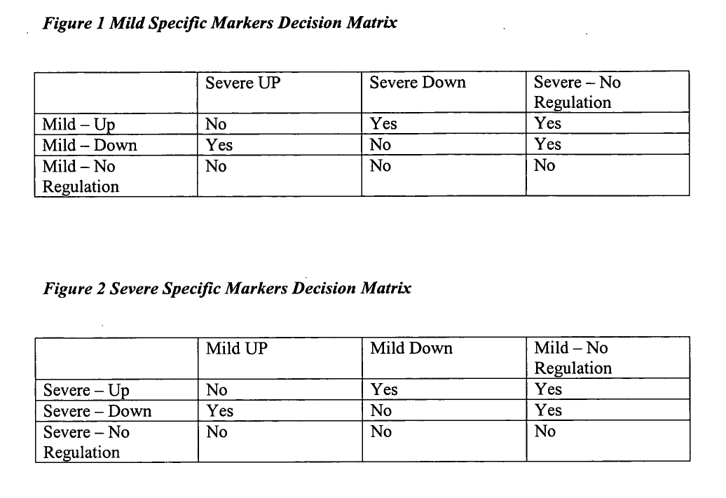

Table 3—Genes identified as severe Osteoarthritis biomarkers on the basis of the query outlined in the decision matrix of FIG. 2.

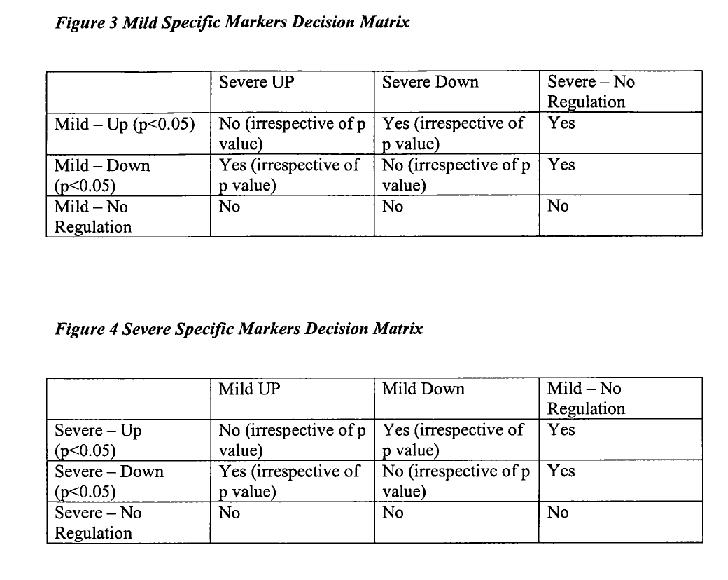

Table 4—Genes identified as a subset of mild Osteoarthritis biomarkers on the basis of the query outlined in the decision matrix of FIG. 3.

Table 5—Genes identified as a subset of severe Osteoarthritis biomarkers on the basis of the query outlined in the decision matrix of FIG. 4.

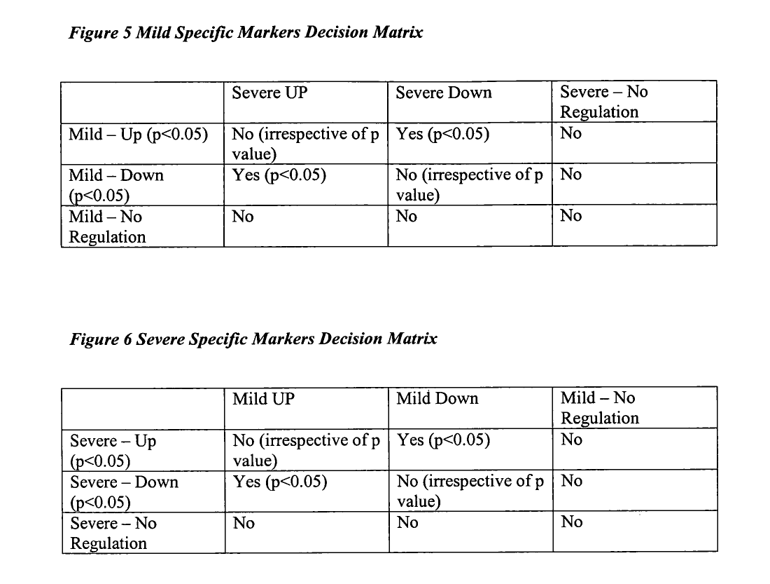

Table 6—Genes identified as a subset of mild Osteoarthritis biomarkers on the basis of the query outlined in the decision matrix of FIG. 5.

Table 7—Genes identified as a subset of severe Osteoarthritis biomarkers on the basis of the query outlined in the decision matrix of FIG. 6.

### DETAILED DESCRIPTION OF THE INVENTION

The invention relates to the identification of OA biomarkers and OA stage specific biomarkers, and to a method of measuring the expression level of the products of the biomarkers so as to diagnose or prognose OA or a specific stage of OA and so as to screen for useful treatments of OA. Further encompassed by the invention is the polynucleotides which specifically and/or selectively hybridize to the product of the biomarkers of the invention to monitor disease progression in an individual and to monitor the efficacy of therapeutic regimens. The invention also identifies targets for use in screening for novel therapeutic compounds useful in treatment of Osteoarthritis in the development of prophylactic and therapeutic compositions for the prevention, treatment, management and/or amelioration of osteoarthritis or a symptom thereof.

Definitions

The following definitions are provided for specific terms which are used in the following written description.

As used herein, “Osteoarthritis” refers to a chronic disease in which the articular cartilage that lies on the ends of bones that form the articulating surface of the joints gradually degenerates over time. Cartilage degeneration can be caused by an imbalanced catabolic activity (removal of “old” cells and matrix components) and anabolic activity (production of “new” cells and molecules) (Westacott et al., 1996, Semin Arthritis Rheum, 25:254-72).

As used herein, “cartilage” or “articular cartilage” refers to elastic, translucent connective tissue in mammals, including human and other species. Cartilage is composed predominantly of chondrocytes, type II collagen, small amounts of other collagen types, other noncollagenous proteins, proteoglycans and water, and is usually surrounded by a perichondrium, made up of fibroblasts, in a matrix of type I and type II collagen as well as other proteoglycans. Although most cartilage becomes bone upon maturation, some cartilage remains in its original form in locations such as the nose, ears, knees, and other joints. The cartilage has no blood or nerve supply and chondrocytes are the only type of cell in this tissue.

As used herein, “chondrocyte” refers to cartilage cells.

As used herein, “synovial fluid” refers to fluid secreted from the “synovial sac” which surrounds each joint. Synovial fluid serves to protect the joint, lubricate the joint and provide nourishment to the articular cartilage. Synovial fluid useful according to the invention contains cells from which RNA can be isolated according to methods well known in the art as described herein.

As used herein, the term “Osteoarthritis (OA) staging” or “Osteoarthritis (OA) grading” refers to determining the degree of advancement or progression of the disease in the cartilage and the “stage of OA” is the characterization of the degree of advancement or progression. In one embodiment, a “stage of OA” includes the absence of the disease. In order to classify cartilage into different disease stages, a scoring system can be used according to known methods in the art. Preferably the scoring system described in Marshall (Marshall W., 1996, The Journal of Rheumatology, 23:582-584, incorporated by reference) is used. According to this method, each of the 6 articular surfaces (patella, femoral trochlea, medial femoral condyle, medial tibial plateau, lateral femoral condyle and lateral tibial plateau) is assigned a cartilage grade based on the worst lesion present on that specific surface. A scoring system is then applied in which each articular surface receives an OA severity number value that reflects the cartilage severity grade for that surface. For example, if the medial femoral condyle has a grade I lesion as its most severe cartilage damage a value of 1 is assigned. A total score for the patient is then derived from the sum of the scores on the 6 articular surfaces. Based on the total score, each patient is placed into one of 4 OA groups: mild (early) (1-6), moderate (7-12), marked (13-18) and severe (>18).

As used herein, the term “biomarker” refers to a gene that is differentially regulated during OA as compared with the gene regulation during non OA. A “biomarker” also refers to a gene that is differentially regulated during the course of OA, for example is differentially regulated as between a specific stage of OA as compared with another stage of OA and/or as compared with the gene regulation during non OA. The differential regulation of a biomarker or a plurality of biomarkers can be monitored by monitoring the products of the biomarker for example through qualitative and/or quantitative analysis of the RNA in the sample of interest. An Osteoarthritis (OA) biomarker or a plurality of biomarkers can be used for a multitude of purposes, including to indicate the presence of OA, a susceptibility or predilection to OA, a specific stage of OA, the progression of OA, the response to treatment or therapy, and determining the efficacy of drugs. A sample of interest can include synovial fluid, cartilage, chondrocytes, blood, lymph, and saliva.

As used herein the term “product of a biomarker” or “RNA product of the biomarker” and “RNA transcripts corresponding to a biomarker” includes “products of stage specific biomarkers” and refers to the RNA expressed by the gene used as a biomarker whose measure of expression can be determined in accordance with methods known in the art and disclosed herein.

As used herein “selective hybridization” or “specific hybridization” in the context of this invention refers to a hybridization which occurs as between a polynucleotides, for example as between a polynucleotide and an RNA product of the biomarker of the invention, wherein the hybridization is such that the polynucleotide specifically binds to the RNA product of the biomarker of the invention. Preferably, the polynucleotide will preferentially bind to the RNA product of the biomarker of the invention with a specificity of greater than 70%, greater than 80%, greater than 90% and most preferably 100% specificity. As would be understood to a person skilled in the art, a polynucleotide which “selectively hybridizes” to the RNA product of a biomarker of the invention can be determined taking into account the length and composition. For example, selective hybridization occurs when two polynucleotide sequences are substantially complementary (at least about 65% complementary over a stretch of at least 14 to 25 nucleotides, preferably at least about 75% complementary, more preferably at least about 90% complementary). See Kanehisa, M., 1984, Nucleic acids Res., 12:203, incorporated herein by reference. As a result, it is expected that a certain degree of mismatch is tolerated. Such mismatch may be small, such as a mono-, di- or tri-nucleotide. Alternatively, a region of mismatch can encompass loops, which are defined as regions in which there exists a mismatch in an uninterrupted series of four or more nucleotides. Numerous factors influence the efficiency and selectivity of hybridization of two nucleic acids, for example, the hybridization of a nucleic acid member on an array to a target nucleic acid sequence. These factors include nucleic acid member length, nucleotide sequence and/or composition, hybridization temperature, buffer composition and potential for steric hindrance in the region to which the nucleic acid member is required to hybridize. A positive correlation exists between the nucleic acid length and both the efficiency and accuracy with which a nucleic acid will anneal to a target sequence. In particular, longer sequences have a higher melting temperature (T_{M}) than do shorter ones, and are less likely to be repeated within a given target sequence, thereby minimizing promiscuous hybridization. Hybridization temperature varies inversely with nucleic acid member annealing efficiency. Similarly the concentration of organic solvents, e.g., formamide, in a hybridization mixture varies inversely with annealing efficiency, while increases in salt concentration in the hybridization mixture facilitate annealing. Under stringent annealing conditions, longer nucleic acids, hybridize more efficiently than do shorter ones, which are sufficient under more permissive conditions. More particularly, for example, the degree of stringency of washing can be varied by changing the temperature, pH, ionic strength, divalent cation concentration, volume and duration of the washing. For example, the stringency of hybridization may be varied by conducting the hybridization at varying temperatures below the melting temperatures of the probes. The melting temperature of the probe may be calculated using the following formulas:

For oligonucleotide probes, between 14 and 70 nucleotides in length, the melting temperature (Tm) in degrees Celcius may be calculated using the formula: Tm=81.5+16.6(log [Na+])+0.41(fraction G+C)−(600/N) where N is the length of the oligonucleotide.

For example, the hybridization temperature may be decreased in increments of 5° C. from 68° C. to 42° C. in a hybridization buffer having a Na+ concentration of approximately 1M. Following hybridization, the filter may be washed with 2×SSC, 0.5% SDS at the temperature of hybridization. These conditions are considered to be “moderate stringency” conditions above 50° C. and “low stringency” conditions below 50° C. A specific example of “moderate stringency” hybridization conditions is when the above hybridization is conducted at 55° C. A specific example of “low stringency” hybridization conditions is when the above hybridization is conducted at 45° C.

If the hybridization is carried out in a solution containing formamide, the melting temperature may be calculated using the equation Tm=81.5+16.6(log [Na⁺])+0.41(fraction G+C)−(0.63% formamide)−(600/N), where N is the length of the probe. For example, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6×SSC, 0.5% SDS at 50° C. These conditions are considered to be “moderate stringency” conditions above 25% formamide and “low stringency” conditions below 25% formamide. A specific example of “moderate stringency” hybridization conditions is when the above hybridization is conducted at 30% formamide. A specific example of “low stringency” hybridization conditions is when the above hybridization is conducted at 10% formamide.

As used herein, the term “stage specific biomarker” includes both “mild specific biomarkers” and “severe specific biomarkers”. A “mild specific biomarker” refers to a gene wherein the gene expression of the gene is distinguishable from gene expression of the same gene in non osteoarthritic cartilage (“normal”) and distinguishable from gene expression in osteoarthritic cartilage from other stages of disease. Gene expression is considered distinguishable if the gene expression in mild osteoarthritic cartilage is measurably different from both the gene expression in non osteoarthritic cartilage and the gene expression in severe osteoarthritic cartilage. As used herein, a “severe Osteoarthritis specific biomarker” refers to a gene wherein the gene expression is distinguishable from gene expression of the same gene in both non osteoarthritic cartilage (“normal”) and in osteoarthritic cartilage from other stages of disease. Gene expression is considered distinguishable if the gene expression in severe osteoarthritic cartilage is measurably different than gene expression in non osteoarthritic cartilage and is measurable different than gene expression in mild osteoarthritic cartilage.

As used herein, the term “significant match”, when referring to nucleic acid sequences, means that two nucleic acid sequences exhibit at least 65% identity, at least 70%, at least 75%, at least 80%, at least 85%, and preferably, at least 90% identity, using comparison methods well known in the art (i.e., Altschul, S. F. et al., 1997, Nucl. Acids Res., 25:3389-3402; Schaffer, A. A. et al., 1999, Bioinformatics 15:1000-1011). As used herein, “significant match” encompasses non-contiguous or scattered identical nucleotides so long as the sequences exhibit at least 65%, and preferably, at least 70%, at least 75%, at least 80%, at least 85%, and preferably, at least 90% identity, when maximally aligned using alignment methods routine in the art.

As herein used, the term “stringent conditions” refers to the hybridization conditions used to allow polynucleotide, polynucleotide interaction. Overall, five factors influence the efficiency and selectivity of hybridization polynucleotide, polynucleotide interaction. These factors, which are (i) polynucleotide (primer) length, (ii) the nucleotide sequence and/or composition, (iii) hybridization temperature, (iv) buffer chemistry and (v) the potential for steric hindrance in the region to which the primer is required to hybridize, are important considerations and contribute to the stringency of the hybridization conditions.

As used herein, the term “level of expression” refers to the measurable quantity of a given nucleic acid as determined by hybridization (relative to a control) or more quantitative measurements such as real-time RT PCR, which includes use of both SYBR® green and TaqMan® technology and which corresponds in direct proportion with the extent to which the gene is expressed. The level of expression of a nucleic acid is determined by methods well known in the art.

As used herein, the term “differentially expressed” or “changes in the level of expression” refers to an increase or decrease in the measurable expression level of a product of the biomarker(s) of the invention in a sample or a population of samples as compared with a control wherein the control can be a sample or a population of samples with a different stage of Osteoarthritis, or a sample or population of samples taken at a different time, or a sample or population of samples pre- or post treatment. As used herein, “differentially expressed” can be measured by comparing the ratio of quantitative data from the test sample with the control. For example, the ratio of the level of expression of a product of a biomarker of the invention in one or a population of test samples as compared with the expression level of the product of the biomarker of the invention in a control where is not equal to 1.0, or in another embodiment the ratio of the quantitative data to the control is increased 1.1 fold, 1.2 fold, 1.5 fold, 2.0 fold, 2.5 fold, 3.0 fold, 4.0 fold, 5.0 fold, 10 fold, 20 fold or more, or in another embodiment the ratio of the quantitative data to the control is decreased 1.1 fold, 1.2 fold, 1.5 fold, 2.0 fold, 2.5 fold, 3.0 fold, 4.0 fold, 5.0 fold, 10 fold, 20 fold or more. A sample is also considered to demonstrate “differential expression” if comparison as between the sample and a control is such that one of the two samples contains no detectable expression of the product of the biomarker. Absolute quantification of the level of expression of a product of a biomarker of the invention can be accomplished by including known concentration(s) of one or more control species, generating a standard curve based on the amount of the control nucleic acid and extrapolating the expression level of the “unknown” species from the standard curve. As used herein “differentially expressed” when referring to EST analysis refers to the relative expression level of a gene based on the frequency of ESTs representing the gene derived from a cDNA library as compared to the frequency of ESTs representing the same gene derived from another cDNA library. As described herein, the “relative EST frequency” of an EST is calculated by dividing the number of ESTs representing each specific gene by the total number of ESTs analyzed. Differences in “relative EST frequency” may be used as an indication of differential gene expression.

For microarray analysis, the level of expression is measured by hybridization analysis using labeled target nucleic acids according to methods well known in the art. The label on the target nucleic acid can be a luminescent label, an enzymatic label, a radioactive label, a chemical label or a physical label. Preferably, target nucleic acids are labeled with a fluorescent molecule. Preferred fluorescent labels include, but are not limited to: fluorescein, amino coumarin acetic acid, tetramethylrhodamine isothiocyanate (TRITC), Texas Red, Cyanine 3 (Cy3) and Cyanine 5 (Cy5).

As used herein, the term “up regulated” or “increased level of expression” in the context of this invention refers to an expressed RNA transcript corresponding to a biomarker wherein the measure of the quantity of the RNA transcript demonstrates an increased level of expression of the gene as compared with the level of an expressed RNA transcript from a different source, sample or group of samples corresponding to the same gene. As used in the context of EST frequency analysis as described herein, “up regulated” is used to describe an increased number of ESTs for a specific gene as between either the mild or severe library and the normal library. As also used herein the term “up regulated” also refers to the comparison as determined using array analysis, quantitative RT-PCR analysis or other similar analysis, in samples isolated from one or more individuals having Osteoarthritis or an identified disease state of Osteoarthritis as determined by Osteoarthritis staging as compared with the same gene in samples isolated from one or more individuals identified as normal with respect to osteoarthritis, or one or more individuals with a different identified disease state of osteoarthritis as determined by osteoarthritis staging. An “increased level of expression” or “up regulated” according to the present invention, is an increase in expression of at least 10% or more, for example, 20%, 30%, 40%, or 50%, 60%, 70%, 80%, 90% or more, or greater than 1-fold, 2-fold, 3-fold, 4-fold, 5-fold, 10-fold, 50-fold, 100-fold or more as measured, for example, by the intensity of hybridization, or as measured by delta Ct value, according to methods of the present invention. For example, up regulated sequences includes sequences having an increased level of expression in cartilage isolated from individuals characterized as having mild, or severe OA as compared with cartilage isolated from normal individuals. Up regulated sequences can also include sequences having an increased level of expression in cartilage from individuals characterized as having one stage of osteoarthritis as compared to another stage of osteoarthritis (e.g. mild OA v. severe OA) As used herein, the term “down regulated” or “decreased level of expression” in the context of this invention refers to an expressed RNA transcript corresponding to a gene wherein the measure of the quantity of the RNA transcript demonstrates a decreased level of expression of the gene as compared with the level of an expressed RNA transcript from a different source, sample or group of samples corresponding to the same gene. As used in the context of EST frequency analysis as described herein, “down regulated” is used to describe a decreased number of ESTs for a specific gene as between either the mild or severe library and the normal library. As also used herein the term “down regulated” also refers to the comparison as determined using array analysis, quantitative RT-PCR analysis or other similar analysis, in cartilage isolated from one or more individuals having osteoarthritis or an identified disease state of osteoarthritis as determined by osteoarthritis staging as compared with the same gene in cartilage isolated from one or more individuals identified as normal with respect to osteoarthritis, or one or more individuals with a different identified disease state of osteoarthritis as determined by osteoarthritis staging. A “decreased level of expression” or “down regulated” according to the present invention, is a decrease in expression of at least 10% or more, for example, 20%, 30%, 40%, or 50%, 60%, 70%, 80%, 90% or more, or less than 1-fold, 2-fold, 3-fold, 4-fold, 5-fold, 10-fold, 50-fold, 100-fold or more as measured, for example, by the intensity of hybridization, or as measured by delta Ct values according to methods of the present invention. For example, up regulated sequences includes sequences having an increased level of expression in cartilage isolated from individuals characterized as having mild, or severe OA as compared with cartilage isolated from normal individuals. Up regulated sequences can also include sequences having an increased level of expression in cartilage from individuals characterized as having one stage of osteoarthritis as compared to another stage of osteoarthritis (e.g. mild OA v. severe OA)

As used herein, the term “not regulated” or “no change in level of expression” or “no regulation” in the context of this invention refers to an expressed RNA transcript corresponding to a gene wherein the measure of the quantity of the RNA transcript demonstrates a similar level of expression of the gene as compared with the level of an expressed RNA transcript from a different source, sample or group of samples corresponding to the same gene. As used in the context of EST frequency analysis as described herein, “no regulation” is used to describe an equivalent number of ESTs as between either the mild or severe library and the normal library. As also used herein the term “no regulation” also refers to the comparison as determined using array analysis, quantitative RT-PCR analysis or other similar analysis, in a sample or group of samples isolated from one or more individuals having osteoarthritis or an identified disease state of osteoarthritis as determined by osteoarthritis staging as compared with the same gene in cartilage isolated from one or more individuals identified as normal with respect to osteoarthritis, or one or more individuals with a different identified disease state of osteoarthritis as determined by osteoarthritis staging.

As used herein, “diagnosis of OA” or “OA diagnosis”, according to the invention, includes determining if an individual is afflicted with OA, or, determining the OA stage or grade of the disease in an individual. In one embodiment “diagnosis of OA” refers to the process of using traditional methods to determine whether a patient has OA or a stage of OA based on the medical history and physical examination of the patient using methods known in the art (i.e., joint X ray). In another embodiment, OA stages are measured using the scoring system described by Marshall, supra. “Prognosis of OA” refers to a prediction of the probable occurrence and/or progression of OA in a patient, as well as the likelihood of recovery from OA, or the likelihood of ameliorating symptoms of OA or the likelihood of reversing the effects of OA. As used herein when referring to biomarkers of the invention, “Diagnosis”, “Diagnosis of OA” or “OA diagnosis”, includes the ability to discriminate between an individual having OA and an individual not having OA and includes the ability to discriminate between the stages of OA. In one embodiment, it refers to the ability to determine whether an individual has a specific stage of OA. “Prognosis of OA” refers to the process of determining whether a person has an increased likelihood of having osteoarthritis, including having an increased likelihood of developing osteoarthritis, an increased likelihood of progressing to a specific stage of OA or of developing symptoms of OA. “Diagnosis” “diagnosis of OA” or “OA diagnosis” also refers to a determination of an increased likelihood that an individual has osteoarthritis, or a specific stage of osteoarthritis (“true positive”) or does not have osteoarthritis or a specific stage of osteoarthritis (“true negative”) while minimizing the likelihood that the individual is improperly characterized as having osteoarthritis or a specific stage of osteoarthritis (“false positive”) or improperly characterized as not having osteoarthritis or said stage of osteoarthritis (“false negative”).

As used herein, “patient” or “individual” refers to a mammal who is diagnosed with arthritis and further includes a mammal who is diagnosed with the mild, moderate, marked, or severe form of OA.

As used herein, a “gene expression pattern” or “gene expression profile” comprises the pattern (i.e., qualitatively and/or quantitatively) of two or more expressed nucleic acid sequences corresponding to two or more genes of the invention. In one embodiment, a “gene expression pattern” or “gene expression profile” refers to a pattern of gene expression calls as up regulated, down regulated or not regulated as compared with a control wherein said control can be a sample or data representing one or more individuals without osteoarthritis, one or more individuals with osteoarthritis, or one or more individuals with a specific stage of osteoarthritis as determined for each individual gene in a group of genes. The “gene expression pattern” or “gene expression profile” can be determined using techniques such as microarray hybridization in combination with GeneSpring™ analysis. The “gene expression pattern” or “gene expression profile” can also be determined using other techniques known in the art to quantitate gene expression so as to allow a determination of up regulation, down regulation or not regulated, such as, for example, quantitative RT-PCR (“QRT-PCR”). As used herein, “a nucleic acid array expression profile” is generated from the hybridization of nucleic acids derived from a sample to one or more nucleic acid members comprising an array according to the invention.

As used herein, a “gene expression pattern” which is “indicative of disease”, or “indicative of Ostreoarthritis”, “indicative of a stage of Osteoarthritis” or used to “diagnose Ostreoarthritis” or to “diagnose a stage of Ostreoarthritis” refers to an expression pattern which is diagnostic of Osteoarthritis or a stage of Osteoarthritis. Preferably the expression pattern is found significantly more often in patients with a disease than in patients without the disease (as determined using routine statistical methods). More preferably, an expression pattern which is indicative of disease is found in at least 70% of patients who have the disease and is found in less than 10% of patients who do not have the disease. Even more preferably, an expression pattern which is indicative of disease is found in at least 75%, at least 80%, at least 85%, at least 90%, at least 95% or more in patients who have the disease and is found in less than 10%, less than 8%, less than 5%, less than 2.5%, or less than 1% of patients who do not have the disease.

As used herein, the “5′ end” refers to the end of an mRNA up to the first 1000 nucleotides or ⅓ of the mRNA (where the full length of the mRNA does not include the poly A tail), starting at the first nucleotide of the mRNA. The “5′ region” of a gene refers to a polynucleotide (double-stranded or single-stranded) located within or at the 5′ end of a gene, and includes, but is not limited to, the 5′ untranslated region, if that is present, and the 5′ protein coding region of a gene. The 5′ region is not shorter than 8 nucleotides in length and not longer than 1000 nucleotides in length. Other possible lengths of the 5′ region include but are not limited to 10, 20, 25, 50, 100, 200, 400, and 500 nucleotides.

As used herein, the “3′ end” refers to the end of an mRNA up to the last 1000 nucleotides or ⅓ of the mRNA, where the 3′ terminal nucleotide is that terminal nucleotide of the coding or untranslated region that adjoins the poly-A tail, if one is present. That is, the 3′ end of an mRNA does not include the poly-A tail, if one is present. The “3′ region” of a gene refers to a polynucleotide (double-stranded or single-stranded) located within or at the 3′ end of a gene, and includes, but is not limited to, the 3′ untranslated region, if that is present, and the 3′ protein coding region of a gene. The 3′ region is not shorter than 8 nucleotides in length and not longer than 1000 nucleotides in length. Other possible lengths of the 3′ region include but are not limited to 10, 20, 25, 50, 100, 200, 400, and 500 nucleotides. As used herein, the “internal coding region” of a gene refers to a polynucleotide (double-stranded or single-stranded) located between the 5′ region and the 3′ region of a gene as defined herein. The “internal coding region” is not shorter than 8 nucleotides in length and not longer than 1000 nucleotides in length. Other possible lengths of the “internal coding region” include but are not limited to 10, 20, 25, 50, 100, 200, 400, and 500 nucleotides.

The 5′, 3′ and internal regions are non-overlapping and may, but need not be contiguous, and may, but need not, add up to the full length of the corresponding gene.

As used herein, the term “oligonucleotide” is defined as a molecule comprised of two or more deoxyribonucleotides and/or ribonucleotides, and preferably more than three. Its exact size will depend upon many factors which, in turn, depend upon the ultimate function and use of the oligonucleotide. The oligonucleotides may be from about 8 to about 1,000 nucleotides long. Although oligonucleotides of 8 to 100 nucleotides are useful in the invention, preferred oligonucleotides range from about 8 to about 15 bases in length, from about 8 to about 20 bases in length, from about 8 to about 25 bases in length, from about 8 to about 30 bases in length, from about 8 to about 40 bases in length or from about 8 to about 50 bases in length.

The term, “primer”, as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the oligonucleotide primer typically contains 15-25 or more nucleotides, although it may contain fewer nucleotides. The factors involved in determining the appropriate length of primer are readily known to one of ordinary skill in the art.

As used herein, the term “probe” means oligonucleotides and analogs thereof and refers to a range of chemical species that recognize polynucleotide target sequences through hydrogen bonding interactions with the nucleotide bases of the target sequences. The probe or the target sequences may be single- or double-stranded RNA or single- or double-stranded DNA or a combination of DNA and RNA bases. A probe is at least 8 nucleotides in length and less than the length of a complete gene. A probe may be 10, 20, 30, 50, 75, 100, 150, 200, 250, 400, 500 and up to 2000 nucleotides in length as long as it is less the full length of the target gene. Probes can include oligonucleotides modified so as to have a tag which is detectable by fluorescence, chemiluminescence and the like. The probe can also be modified so as to have both a detectable tag and a quencher molecule, for example Taqman® and Molecular Beacon® probes.

The oligonucleotides and analogs thereof may be RNA or DNA, or analogs of RNA or DNA, commonly referred to as antisense oligomers or antisense oligonucleotides. Such RNA or DNA analogs comprise but are not limited to 2-′O-alkyl sugar modifications, methylphosphonate, phosphorothiate, phosphorodithioate, formacetal, 3′-thioformacetal, sulfone, sulfamate, and nitroxide backbone modifications, and analogs wherein the base moieties have been modified. In addition, analogs of oligomers may be polymers in which the sugar moiety has been modified or replaced by another suitable moiety, resulting in polymers which include, but are not limited to, morpholino analogs and peptide nucleic acid (PNA) analogs (Egholm, et al. Peptide Nucleic Acids (PNA)-Oligonucleotide Analogues with an Achiral Peptide Backbone, (1992)).

Probes may also be mixtures of any of the oligonucleotide analog types together or in combination with native DNA or RNA. At the same time, the oligonucleotides and analogs thereof may be used alone or in combination with one or more additional oligonucleotides or analogs thereof.

As used herein, a “nucleic acid target” or a “nucleic acid marker” or a “nucleic acid member on an array”” also includes nucleic acid immobilized on an array and capable of binding to a nucleic acid member of complementary sequence through sets of non-covalent bonding interactions, including complementary base pairing interactions. As used herein, a nucleic acid target may include natural (i.e., A, G, C, or T) or modified bases (7-deazaguanosine, inosine, etc.). In addition, the bases in nucleic acid probes may be joined by a linkage other than a phosphodiester bond, so long as it does not interfere with hybridization (i.e., the nucleic acid target still specifically binds to its complementary sequence under standard stringent or selective hybridization conditions). Thus, nucleic acid targets may be peptide nucleic acids in which the constituent bases are joined by peptide bonds rather than phosphodiester linkages.

As used herein, “normal” refers to one or more individuals who have not shown any OA symptoms has and/or have not been diagnosed with cartilage injury or OA. “Normal”, according to the invention, also refers to a sample taken from a normal individual within 14 hours post-mortem. A normal cartilage tissue sample, for example, refers to the whole or a piece of cartilage isolated from cartilage tissue within 14 hours post-mortem from an individual who was not diagnosed with OA and whose corpse does not show any symptoms of OA at the time of tissue removal. In alternative embodiments of the invention, the “normal” cartilage tissue sample is isolated from cartilage tissue less than 14 hours post-mortem, e.g., within 13 hours, 12 hours, 11 hours, 10 hours, 9 hours, 8 hours, 7 hours, 6 hours, 5 hours, 4 hours, 3 hours, 2 hours, or 1 hour post-mortem. In one embodiment of the invention, the “normal” cartilage sample is isolated at 14 hours post-mortem and the integrity of mRNA samples extracted is confirmed.

As used herein, “mRNA integrity” refers to the quality of mRNA extracts from samples. mRNA extracts with good integrity do not appear to be degraded when examined by methods well known in the art, for example, by RNA agarose gel electrophoresis (e.g., Ausubel et al., John Weley & Sons, Inc., 1997, Current Protocols in Molecular Biology). Preferably, the mRNA samples have good integrity (e.g., less than 10%, preferably, less than 5%, and more preferably, less than 1% of the mRNA is degraded) to truly represent the gene expression levels of the cartilage samples from which they are extracted.

As used herein, “nucleic acid(s)” or “polynucleotide” generally refers to any polyribonucleotide or poly-deoxyribonucleotide, which may be unmodified RNA or DNA or modified RNA or DNA. “Nucleic acids” include, without limitation, single- and double-stranded nucleic acids. As used herein, the term “nucleic acid(s)” also includes DNAs or RNAs as described above, that contain one or more modified bases. Thus, DNAs or RNAs with backbones modified for stability or for other reasons are “nucleic acids”. The term “nucleic acids” as it is used herein embraces such chemically, enzymatically or metabolically modified forms of nucleic acids, as well as the chemical forms of DNA and RNA characteristic of viruses and cells, including for example, simple and complex cells. A “nucleic acid” or “nucleic acid sequence” may also be an expressed sequence tag (EST) according to some embodiments of the invention. An EST is a small part of the expressed sequence of a gene (i.e., the “tag” of a sequence), made from cDNA. An EST can be used to fish the rest of the gene out of the chromosome, by matching base pairs with part of the expressed sequence of the gene.

As used herein, “isolated” or “purified” when used in reference to a nucleic acid means that a naturally occurring sequence has been removed from its normal cellular (e.g., chromosomal) environment or is synthesized in a non-natural environment (e.g., artificially synthesized). Thus, an “isolated” or “purified” sequence may be in a cell-free solution or placed in a different cellular environment. The term “purified” does not imply that the sequence is the only nucleotide present, but that it is essentially free (about 90-95% pure) of non-nucleotide material naturally associated with it, and thus is distinguished from isolated chromosomes.

As defined herein, a “nucleic acid array” refers a plurality of unique nucleic acids (or “nucleic acid members”) attached to a support at a density exceeding 20 different nucleic acids/cm² where each of the nucleic acid members is attached to a surface of a support in a non-identical pre-selected region. In one embodiment, the nucleic acid member attached to the surface of a support is DNA. In a preferred embodiment, the nucleic acid member attached to the surface of a support is cDNA. In another preferred embodiment, the nucleic acid member attached to the surface of a support is cDNA synthesized by polymerase chain reaction (PCR). Preferably, a nucleic acid member of the array according to the invention is at least 50 nucleotides in length. Preferably, a nucleic acid member of the array is less than 6,000 nucleotides in length. More preferably, a nucleic acid member of the array comprises an array less than 500 nucleotides in length. In one embodiment, the array comprises at least 500 different nucleic acid members attached to one surface of the solid support. In another embodiment, the array comprises at least 10 different nucleic acid members attached to one surface of the solid support. In yet another embodiment, the array comprises at least 10,000 different nucleic acid members attached to one surface of the solid support. In yet another embodiment, the array comprises at least 15,000 different nucleic acid members attached to one surface of the solid support. The term “nucleic acid”, as used herein, is interchangeable with the term “nucleic acid”.

As used herein, “a plurality of” or “a set of” refers to more than two, for example, 3 or more, 100 or more, or 1000 or more.

As used herein, “attaching” or “spotting” refers to a process of depositing a nucleic acid target or member onto a substrate to form a nucleic acid array such that the nucleic acid is irreversibly bound to the substrate via covalent bonds, hydrogen bonds or ionic interactions.

As used herein, “stably associated” refers to a nucleic acid that is irreversibly bound to a substrate to form an array via covalent bonds, hydrogen bonds or ionic interactions such that the nucleic acid retains its unique pre-selected position relative to all other nucleic acids that are stably associated with an array, or to all other pre-selected regions on the substrate under conditions in which an array is typically analyzed (i.e., during one or more steps of hybridization, washes, and/or scanning, etc.).

As used herein, “substrate” or “support” refers to a material having a surface and used to array nucleic acids. The terms “substrate” and “support” are used interchangeably herein. The support may be biological, non-biological, organic, inorganic, or a combination of any of these, existing as particles, strands, precipitates, gels, sheets, tubing, spheres, beads, containers, capillaries, pads, slices, films, plates, slides, chips, etc. Often, the substrate is a silicon or glass surface, (poly)tetrafluoroethylene, (poly)vinylidendifluoride, polystyrene, polycarbonate, a charged membrane, such as nylon 66 or nitrocellulose, or combinations thereof. In a preferred embodiment, the support is glass. Preferably, the surface of the support will contain reactive groups, including, but not limited to, carboxyl, amino, hydroxyl, thiol, and the like. In one embodiment, the surface is optically transparent. For example, supports included in this context includes supports which are two-dimensional or three-dimensional such as the microarray support described in U.S. Pat. No. 5,843,767.

As used herein, “pre-selected region”, “predefined region”, or “unique position” refers to a localized area on a substrate which is, was, or is intended to be used for the deposit of a nucleic acid member and is otherwise referred to herein in the alternative as a “selected region” or simply a “region.” The pre-selected region may have any convenient shape, e.g., circular, rectangular, elliptical, wedge-shaped, etc. In some embodiments, a pre-selected region is smaller than about 1 cm², more preferably less than 1 mm², still more preferably less than 0.5 mm², and in some embodiments less than 0.1 mm². A nucleic acid member at a “pre-selected region”, “predefined region”, or “unique position” is one whose identity (e.g., sequence) can be determined by virtue of its position at the region or unique position.

As used herein, a “cartilage nucleic acid sample”, refers to nucleic acids derived from cartilage. Preferably, a cartilage nucleic acid sample is RNA or is a nucleic acid corresponding to RNA, for example, cDNA.

As used herein, the term “hybridizing to” or “hybridization” refers to the hydrogen binding with a complementary nucleic acid, via an interaction between for example, a target nucleic acid sequence and a nucleic acid member in an array. Hybridization methods for nucleic acids are well known to those of ordinary skill in the art (see, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York). The nucleic acid molecules from an osteoarthritis sample hybridize under stringent conditions to nucleic acid biomarkers expressed in osteoarthritis. In one embodiment the biomarkers are sets of one or two or more of the nucleic acid molecules derived from the sets of genes listed in Tables 1, 2, 3, 4, 5, 6, and/or 7. Also encompassed by the invention are variants of those genes, such as allelic variants or single nucleotide polymorphisms (SNPs) in tissues. Accordingly, methods for identifying osteoarthritis nucleic acid biomarkers, including variants of the disclosed full-length cDNAs, genomic DNAs, and SNPs are also included in the invention.

As used herein, the term “significant match”, when referring to nucleic acid sequences, means that two nucleic acid sequences exhibit at least 65% identity, at least 70%, at least 75%, at least 80%, at least 85%, and preferably, at least 90% identity, using comparison methods well known in the art (i.e., Altschul, S. F. et al., 1997, Nucl. Acids Res., 25:3389-3402; Schaffer, A. A. et al., 1999, Bioinformatics 15:1000-1011). As used herein, “significant match” encompasses non-contiguous or scattered identical nucleotides so long as the sequences exhibit at least 65%, and preferably, at least 70%, at least 75%, at least 80%, at least 85%, and preferably, at least 90% identity, when maximally aligned using alignment methods routine in the art.

As used herein, a “novel sequence” or “novel expressed sequence tag (EST)” refers to a nucleic acid sequence which has no significant match to any existing sequence in the “nt”, “nr”, “est”, “gss” and “htg” databases available through NCBI at the time each novel sequence was compared. “No significant match” preferably refers to a less than 65% match between a novel sequence being queried against other sequences in the database, and preferably, a less than 50% match, a less than 40% match, or a less than 30% match, after maximally aligning sequences using methods routine in the art.

As used herein, a “known sequence” refers to a nucleic acid sequence which has significant match to at least one existing sequence in the “nt”, “nr”, “est”, “gss” and “htg” databases available through NCBI. “Known sequence with a function” refers to a nucleic acid with significant match to an existing sequence which encodes a polypeptide with a known function. “Known sequence with no function” refers to a nucleic acid that exhibits a significant match to an existing sequence which encodes a polypeptide of unknown function.

As used herein, a “chondrocyte-specific nucleic acid” is a nucleic acid sequence which is expressed at a detectable level in a chondrocyte and is not expressed at a detectable level in any other cell types as indicated by having no significant match to any sequence in any of the available databases comprising sequences from other cell types.

As used herein, a “chondrocyte enriched nucleic acid” or “chondrocyte enriched sequence” refers to a sequence which is differentially expressed in chondrocytes as compared to non-chondrocytes.

As used herein, a “subject” or “patient” or “individual” is a human, non-human primate, cow, horse, pig, sheep, goat, dog, cat, or rodent. In all embodiments human subjects are preferred.

Identifying Osteoarthritis Biomarkers

cDNA libraries were constructed from human normal, mild osteoarthritic and severe osteoarthritic cartilage samples. The known and novel clones derived from these libraries were then used to construct human chondrocyte-specific microarrays to generate differential gene expression profiles useful as a diagnostic tool for detection of osteoarthritis. Biomarkers of the invention are useful as a gold standard for osteoarthritis diagnosis and for use to identify and monitor therapeutic efficacy of new drug targets.

One effective and rapid way of characterizing gene expression patterns in a given tissue is through large-scale partial sequencing of a cDNA library produced from such a tissue to generate expressed sequence tags (ESTs). This approach has provided both quantitative and qualitative information on gene expression in a variety of tissues and cells (4-7). Since cDNA libraries represent gene transcription in the cells of the tissue used to construct the library, gene expression profiles generated by random sampling and sequencing is used for detailed genetic-level comparison between developmental, normal and pathological states of the tissue examined.

Many human genes are expressed at different levels in cartilage of different disease states. In some cases, a gene is not expressed at all in some disease states, and at high levels in others. According to the invention, differential analysis of chondrocyte gene expression during different stages of cartilage disease using an EST-based approach has identified genes that play important roles in osteoarthritis pathogenesis and cartilage repair. The advantage of this method is that it provides gene expression information on a larger scale than other methods. This type of genomic-based approach has provided important novel insights the osteoarthritis disease process and provides for novel diagnostic, prognostic and therapeutic approaches.

Samples

Cartilage

In one aspect, cartilage is obtained from a fetus using methods known in the art. The chondrocytes of fetal cartilage have a higher level of metabolic activity and cell division rates as compared to chondrocytes from cartilage from either a normal adult or from an individual diagnosed with any stage of osteoarthritis (such as mild and severe).

In another aspect, cartilage is obtained from a normal individual who is alive or is obtained from cartilage tissue less than 14 hours post mortem, according to methods known in the art and described below. Normal articular cartilage from human adults are obtained using any known method. However, truly normal cartilage cannot generally be sampled from live donors due to ethical considerations. Preferably, normal cartilage samples are obtained from deceased donors, within a fourteen-hour post-mortem window, after cessation of perfusion to the sampled joint, minimize the degradation of RNA observed beyond the window. In other embodiments, the “normal” tissue is obtained less than 14 hours post-mortem, such as 13, 12, 11, 10, 9, 8, 6, 4, 2 or 1 hour post-mortem. A baboon study was conducted to confirm this approach and is described herein below in Example 10. Preferably the normal cartilage is obtained less than 14 hours post-mortem. More preferably, the normal cartilage is obtained less than 12 hours post-mortem.

Preferably, cartilage also is isolated from the following disease stages of osteoarthritis: mild, marked, moderate and severe. Human cartilage samples from osteoarthritic individuals are obtained using any known method. Preferably the cartilage is obtained from individuals undergoing arthroscopy or total knee replacements and samples are stored in liquid nitrogen until needed. In a preferred embodiment, a minimum of 0.05 g of cartilage sample is isolated to obtain 2 □g total RNA extract for the construction of a cDNA library. In another preferred embodiment, a minimum of 0.025 g cartilage sample is isolated to obtain 1 □g total RNA extract to use as a target sample for a microarray. A cartilage sample that is useful according to the invention is in an amount that is sufficient for the detection of one or more polynucleotide sequences according to the invention.

Blood and Synovial Fluid

Samples useful according to the invention also include blood and synovial fluid samples.

In one aspect, blood is obtained from a normal patient or from an individual diagnosed with, or suspected of having, osteoarthritis according to methods of phlebotomy well known in the art. A blood sample useful according to the invention is in an amount ranging from 1 μl to 100 ml, preferably 10 μl to 50 ml, more preferably 10 μl to 25 ml and most preferably 10 μl to 1 ml. A blood sample that is useful according to the invention is in an amount that is sufficient for the detection of one or more nucleic acid sequences according to the invention. In one embodiment, nucleic acids contained within the blood sample are amplified, for example, by polymerase chain reaction (PCR) or by RT-PCR. Other amplification methods known in the art are also encompassed within the scope of the invention (e.g., ligase chain reaction, NASBA, 3SR, and the like).

A synovial fluid sample is obtained from an individual diagnosed with, or suspected of having osteoarthritis according to methods well known in the art. Preferably, synovial fluid is collected from a human knee joint by aspiration at arthroscopy. A synovial fluid sample useful according to the invention is in an amount ranging from 0.1 ml to 20 ml and preferably 0.5 ml to 10 ml. A synovial fluid sample that is useful according to the invention is in an amount that is sufficient for the detection of one or more nucleic acid sequences according to the invention.

Disease Stages of Articular Cartilage

Chondrocytes are preferably obtained from any of the following developmental and disease stages: normal, mild osteoarthritic, moderate osteoarthritic, marked osteoarthritic or severe osteoarthritic.

Cartilage isolated from a “normal” individual, defined herein, also is useful according to the invention for isolation and analysis of “normal” chondrocytes.

Cartilage isolated from a patient diagnosed with any one of: mild and severe osteoarthritis also is useful in the present invention.

In order to classify cartilage according to disease state, a scoring system is used, whereby subjective decisions by the arthroscopist are minimized. The scoring system which defines disease states described herein is that of Marshall, supra, incorporated herein by reference. According to this method, each of the 6 articular surfaces (patella, femoral trochlea, medial femoral condyle, medial tibial plateau, lateral femoral condyle and lateral tibial plateau) is assigned a cartilage grade based on the worst lesion present on that specific surface. A scoring system is then applied in which each articular surface receives an osteoarthritis severity number value that reflects the cartilage severity grade for that surface, as described in Table 9 below.

TABLE 9
Articular Cartilage Grading System

| Grade | Articular Cartilage | Points |
|---|---|---|
| 0 | Normal | 0 |
| I | Surface intact-softening, edema | 1 |
| II | Surface-disrupted-partial thickness lesions | 2 |
| | (no extension to bone) | |
| III | Full thickness lesions-extensions to intact | 3 |
| | bone | |
| IV | Bone erosion or eburnation | 4 |

For example, if the medial femoral condyle has a grade I lesion as its most severe cartilage damage a value of 1 is assigned. A total score for the patient is then derived from the sum of the scores of the 6 articular surfaces. Based on the total score, each patient is placed into one of 4 osteoarthritis groups: mild (1-6), moderate (7-12), marked (13-18) and severe (>18).

RNA Preparation

In one aspect, RNA is isolated from cartilage samples from various disease or developmental stages as described herein. Samples can be from single patients or can be pooled from multiple patients.

In another aspect, RNA is isolated directly from synovial fluid of persons with various disease or developmental stages of osteoarthritis as described herein. Samples can be from single patients or can be pooled from multiple patients.

In another aspect, RNA is isolated directly from blood samples of persons with various disease or developmental stages of osteoarthritis as described herein. Samples can be from single patients or can be pooled from multiple patients.

Total RNA is extracted from the cartilage samples according to methods well known in the art. In one embodiment, RNA is purified from cartilage tissue according to the following method. Following removal of a tissue of interest from an individual or patient, the tissue is quick frozen in liquid nitrogen, to prevent degradation of RNA. Upon the addition of a volume of tissue guanidinium solution, tissue samples are ground in a tissuemizer with two or three 10-second bursts. To prepare tissue guanidinium solution (1 L) 590.8 g guanidinium isothiocyanate is dissolved in approximately 400 ml DEPC-treated H₂O. 25 ml of 2 M Tris-Ci, pH 7.5 (0.05 M final) and 20 ml Na₂EDTA (0.01 M final) is added, the solution is stirred overnight, the volume is adjusted to 950 ml, and 50 ml 2-ME is added.

Homogenized tissue samples are subjected to centrifugation for 10 min at 12,000×g at 12° C. The resulting supernatant is incubated for 2 min at 65° C. in the presence of 0.1 volume of 20% Sarkosyl, layered over 9 ml of a 5.7M CsCl solution (0.1 g CsCl/ml), and separated by centrifugation overnight at 113,000×g at 22° C. After careful removal of the supernatant, the tube is inverted and drained. The bottom of the tube (containing the RNA pellet) is placed in a 50 ml plastic tube and incubated overnight (or longer) at 4° C. in the presence of 3 ml tissue resuspension buffer (5 mM EDTA, 0.5% (v/v) Sarkosyl, 5% (v/v) 2-ME) to allow complete resuspension of the RNA pellet. The resulting RNA solution is extracted sequentially with 25:24:1 phenol/chloroform/isoamyl alcohol, followed by 24:1 chloroform/isoamyl alcohol, precipitated by the addition of 3 M sodium acetate, pH 5.2, and 2.5 volumes of 100% ethanol, and resuspended in DEPC water (Chirgwin et al., 1979, Biochemistry, 18:5294).

Alternatively, RNA is isolated from cartilage tissue according to the following single step protocol. The tissue of interest is prepared by homogenization in a glass teflon homogenizer in 1 ml denaturing solution (4M guanidinium thiosulfate, 25 mM sodium citrate, pH 7.0, 0.1M 2-ME, 0.5% (w/v) N-laurylsarkosine) per 100 mg tissue. Following transfer of the homogenate to a 5-ml polypropylene tube, 0.1 ml of 2 M sodium acetate, pH 4, 1 ml water-saturated phenol, and 0.2 ml of 49:1 chloroform/isoamyl alcohol are added sequentially. The sample is mixed after the addition of each component, and incubated for 15 min at 0-4° C. after all components have been added. The sample is separated by centrifugation for 20 min at 10,000×g, 4° C., precipitated by the addition of 1 ml of 100% isopropanol, incubated for 30 minutes at −20° C. and pelleted by centrifugation for 10 minutes at 10,000×g, 4° C. The resulting RNA pellet is dissolved in 0.3 ml denaturing solution, transferred to a microfuge tube, precipitated by the addition of 0.3 ml of 100% isopropanol for 30 minutes at −20° C., and centrifuged for 10 minutes at 10,000×g at 4° C. The RNA pellet is washed in 70% ethanol, dried, and resuspended in 100-2001 μl DEPC-treated water or DEPC-treated 0.5% SDS (Chomczynski and Sacchi, 1987, Anal. Biochem., 162:156).

Preferably, the cartilage samples are finely powdered under liquid nitrogen and total RNA is extracted using TRIzol® reagent (GIBCO/BRL).

Alternatively, RNA is isolated from blood by the following protocol. Lysis Buffer is added to blood sample in a ratio of 3 parts Lysis Buffer to 1 part blood (Lysis Buffer (IL) 0.6 g EDTA; 1.0 g KHCO₂, 8.2 g NH₄Cl adjusted to pH 7.4 (using NaOH)). Sample is mixed and placed on ice for 5-10 minutes until transparent. Lysed sample is centrifuged at 1000 rpm for 10 minutes at 4° C., and supernatant is aspirated. Pellet is resuspended in 5 ml Lysis Buffer, and centrifuged again at 1000 rpm for 10 minutes at 4° C. Pelleted cells are homogenized using TRIzol® (GIBCO/BRL) in a ratio of approximately 6 ml of TRIzol® for every 10 ml of the original blood sample and vortexed well. Samples are left for 5 minutes at room temperature. RNA is extracted using 1.2 ml of chloroform per 1 ml of TRIzol®. Sample is centrifuged at 12,000×g for 5 minutes at 4° C. and upper layer is collected. To upper layer, isopropanol is added in ratio of 0.5 ml per 1 ml of TRIzol®. Sample is left overnight at −20° C. or for one hour at −20° C. RNA is pelleted in accordance with known methods, RNA pellet air dried, and pellet resuspended in DEPC treated ddH₂O. RNA samples can also be stored in 75% ethanol where the samples are stable at room temperature for transportation.

Alternatively, RNA is isolated from synovial fluid using TRIzol® reagent (GIBCO/BRL).

Purity and integrity of RNA is assessed by absorbance at 260/280 nm and agarose gel electrophoresis followed by inspection under ultraviolet light.

Construction of cDNA Libraries

cDNA libraries are constructed according to methods well known in the art (see for example Ausubel, supra, and Sambrook, supra, incorporated herein by reference).

In one aspect, cDNA samples, i.e., DNA that is complementary to RNA such as mRNA are prepared. The preparation of cDNA is well-known and well-documented in the prior art.

cDNA may be prepared according to the following method. Total cellular RNA is isolated (as described) and passed through a column of oligo(dT)-cellulose to isolate polyA RNA. The bound polyA mRNAs are eluted from the column with a low ionic strength buffer. To produce cDNA molecules, short deoxythymidine oligonucleotides (12-20 nucleotides) are hybridized to the polyA tails to be used as primers for reverse transcriptase, an enzyme that uses RNA as a template for DNA synthesis. Alternatively, or additionally, mRNA species are primed from many positions by using short oligonucleotide fragments comprising numerous sequences complementary to the mRNA of interest as primers for cDNA synthesis. The resultant RNA-DNA hybrid is converted to a double stranded DNA molecule by a variety of enzymatic steps well-known in the art (Watson et al., 1992, Recombinant DNA, 2nd edition, Scientific American Books, New York).

To construct a cDNA library, the poly (A)⁺ RNA fraction may be isolated by oligo-dT cellulose chromatography (Pharmacia), and 3-5 μg poly (A)⁺ RNA is used to construct a cDNA library in the λ ZAP Express vector (Stratagene). Alternatively, cDNA libraries may be constructed into λTriplEx2 vector through a PCR-based method, using SMART (Switching Mechanism At 5′ end of RNA Transcript) cDNA Library Construction Kit (Clontech). First-strand cDNA is synthesized with an Xho I-oligo (dT) adapter-primer in the presence of 5′-methyl dCTP. After second-strand synthesis and ligation of EcoRI adapters, the cDNAs are digested with Xho I, resulting in cDNA flanked by EcoRI sites at the 5′-ends and Xho I sites at the 3′-ends. Digested cDNAs are size-fractionated in Sephacryl S-500 spin columns (Stratagene), then ligated into the λ ZAP Express vector predigested with EcoRI and Xho I. The resulting DNA/cDNA concatomers are packaged using Gigapack Gold packaging extracts. After titration, aliquots of primary packaging mix are stored in 7% DMSO at −80° C. as primary library stocks, and the rest are amplified to establish stable library stocks.

From the amplified library, phage plaques are plated onto an appropriate medium. Preferably, phage plaques are plated at a density of 200-500 pfu/150 mm plate onto an Escherichia coli XL1-blue MRF′ lawn with IPTG/X-gal for color selection. The plaques are then randomly picked and positive inserts are identified by polymerase chain reaction (PCR), according to methods well known in the art and described hereinbelow. Preferably, plaques are picked into 75 μl suspension media buffer (100 mM NaCl, 10 mM MgSO₄, 1 mM Tris, pH7.5, 0.02% gelatin). Phage elutes (5 μl) may be used for PCR reactions (50 μltotal volume) with 125 μmol/L of each dNTP (Pharmacia), 10 pmol each of modified T3 (5′-GCCAAGCTCGAAATTAACCCTCACTAAAGGG-3′) (SEQ ID NO:1) and T7 (5′-CCAGTGAATTGTAATACGACTCACTATAGGGCG-3′) (SEQ ID NO:2) primers, and 2 U of Taq DNA polymerase (Pharmacia). Reactions are cycled in a DNA Thermal Cycler (Perkin-Elmer) [denaturation at 95° C. for 5 minutes, followed by 30 cycles of amplification (94° C., 45 seconds; 55° C., 30 seconds; 72° C., 3 minutes) and a terminal isothermal extension (72° C., 3 minutes)]. Agarose gel electrophoresis is used to assess the presence and purity of inserts.

The PCR product is then subjected to DNA sequencing using known methods (see Ausubel et al., supra and Sambrook et al., supra). Methods of sequencing employ such enzymes as the Klenow fragment of DNA polymerase I, Sequenase® (US Biochemical Corp, Cleveland, Ohio), Taq polymerase (Perkin Elmer, Norwalk, Conn.), thermostable T7 polymerase (Amersham, Chicago, Ill.), or combinations of recombinant polymerases and proofreading exonucleases such as the ELONGASE Amplification System (Gibco BRL, Gaithersburg, Md.). Preferably, the process is automated with machines such as the Hamilton Micro Lab 2200 (Hamilton, Reno Nev.), Peltier Thermal Cycler (PTC200; MJ Research, Watertown, Mass.), the ABI 377 DNA sequencers (Perkin Elmer), and the PE Biosystems ABI Prism 3700 DNA Analyzer.

PCR products are first subjected to DNA sequencing reactions using specific primers, BigDye™ Terminator Cycle Sequencing v2.0 Ready Reaction (PE Biosystems), Tris MgCl buffer and water in a thermocycler. Sequencing reactions were incubated at 94° C. for 2 minutes, followed by 25 cycles of 94° C., 30 seconds; 55° C., 20 seconds; and 72° C., 1 minute; and 15 cycles of 94° C., 30 seconds; and 72° C. for 1 minute; and 72° C. for 5 minutes. Reactions were then put on hold at 4° C. until purified using methods well known in the prior art (i.e. alcohol precipitation or ethanol precipitation). Automated sequencing is preferably carried out with a PE Biosystems ABI Prism 3700 DNA Analyzer.

PCR

In one aspect, nucleic acid sequences of the invention are amplified by the polymerase chain reaction (PCR). PCR methods are well-known to those skilled in the art.

PCR provides a method for rapidly amplifying a particular nucleic acid sequence by using multiple cycles of DNA replication catalyzed by a thermostable, DNA-dependent DNA polymerase to amplify the target sequence of interest. PCR requires the presence of a nucleic acid to be amplified, two single-stranded oligonucleotide primers flanking the sequence to be amplified, a DNA polymerase, deoxyribonucleoside triphosphates, a buffer and salts.

The method of PCR is well known in the art. PCR, is performed as described in Mullis and Faloona, 1987, Methods Enzymol., 155: 335, herein incorporated by reference.

PCR is performed using template DNA (at least 1 fg; more usefully, 1-1000 ng) and at least 25 pmol of oligonucleotide primers. A typical reaction mixture includes: 2111 of DNA, 25 pmol of oligonucleotide primer, 2.5 μl of 10×PCR buffer 1 (Perkin-Elmer, Foster City, Calif.), 0.4 μl of 1.25 μM dNTP, 0.15 μl (or 2.5 units) of Taq DNA polymerase (Perkin Elmer, Foster City, Calif.) and deionized water to a total volume of 25 μl. Mineral oil is overlaid and the PCR is performed using a programmable thermal cycler.

The length and temperature of each step of a PCR cycle, as well as the number of cycles, are adjusted according to the stringency requirements in effect. Annealing temperature and timing are determined both by the efficiency with which a primer is expected to anneal to a template and the degree of mismatch that is to be tolerated. The ability to optimize the stringency of primer annealing conditions is well within the knowledge of one of moderate skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1 minute). The final extension step is generally carried out for 4 minutes at 72° C., and may be followed by an indefinite (0-24 hour) step at 4° C.

Several techniques for detecting PCR products quantitatively without electrophoresis may be useful according to the invention. One of these techniques, for which there are commercially available kits such as Taqman™ (Perkin Elmer, Foster City, Calif.), is performed with a transcript-specific antisense probe. This probe is specific for the PCR product (e.g. a nucleic acid fragment derived from a gene) and is prepared with a quencher and fluorescent reporter probe complexed to the 5′ end of the oligonucleotide. Different fluorescent markers are attached to different reporters, allowing for measurement of two products in one reaction. When Taq DNA polymerase is activated, it cleaves off the fluorescent reporters of the probe bound to the template by virtue of its 5′-to-3′ exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions are performed in 96 well plates so that samples derived from many individuals are processed and measured simultaneously. The Taqman™ system has the additional advantage of not requiring gel electrophoresis and allows for quantification when used with a standard curve.

Nucleic Acid Sequences Useful According to the Invention

The invention provides for isolated nucleic acid sequences including ESTs which can be used as probes, arrayed on microarrays, and/or used for the development of therapies to treat osteoarthritis.

In one aspect, cartilage gene expression profiles at different developmental stages are identified. Another aspect of the invention is to monitor cartilage gene expression profiles of osteoarthritis patients diagnosed with different stages of osteoarthritis. A third aspect of the invention is to screen for potential therapeutic agents which alter the gene expression profile of diseased cartilage cells. The invention therefore provides for nucleic acid sequences that are present at each of the following developmental and disease stages: normal, mild osteoarthritic, and severe osteoarthritic. The invention also provides for nucleic acid sequences that are differentially expressed in any two of the following developmental and disease stages: normal, mild osteoarthritic, and severe osteoarthritic.

Nucleic acids useful according to the invention are prepared by isolating cartilage tissue samples from a developmental or disease stage (normal, mild osteoarthritic, and severe osteoarthritic), preparing a cDNA library (as described above), and performing large-scale partial sequencing (described herein) of the cDNA library to generate Expressed Sequence Tags (ESTs). An EST useful according to the invention is preferably in the range of 50-1000 nucleotides and most preferably 100-500 nucleotides in length.

The invention provides for nucleic acid sequences or ESTs that are categorized as “novel” or “known”, including “known sequences with a function” and “known sequences without a known function”, all defined herein.

As used in herein, a comparison is “statistically significant” when there is only a small probability that similar results would have been observed if the tested hypothesis (i.e., the genes are not expressed at different levels) were true. A small probability can be defined as the accepted threshold level at which the results being compared are considered significantly different. In one embodiment, the accepted lower threshold less than 0.05 (i.e., there is a 5% likelihood that the results would be observed between two or more identical populations) such that any values determined by statistical means at or below this threshold are considered significant.

When comparing two or more samples for similarities, (such as in EST frequency) as the statistically significant of the results are indicated by the p value. P value is a measure of the probability that similar results would have been observed if the tested hypothesis (i.e., the genes are not expressed at different levels) were true. A small probability can be defined as the accepted threshold level at which the results being compared are considered significantly different. In one embodiment, the accepted lower threshold less than 0.05 (i.e., there is a 5% likelihood that the results would be observed between two or more identical populations) such that any values determined by statistical means above this threshold are not considered significantly different and thus similar.

Identification of potential differentially expressed genetic biomarkers in chondrocytes from individuals with ostearthritis as compared to individuals without osteoarthritis is determined by statistical analysis of a comparison of the frequency of ESTs from cDNA libraries prepared from chondrocytes of individuals with osteoarthritis (mild or severe) or individuals without osteoarthritis using the Wilcox Mann Whitney rank sum test. Other statistical tests can also be used, see for example (Sokal and Rohlf (1987) Introduction to Biostatistics 2^{nd} edition, WH Freeman, New York), which is incorporated herein in their entirety.

The invention simplifies diagnosis and prognosis by providing an identified set of one or more genes whose expression in osteoarthritis predicts clinical outcome. In one aspect of the invention, RNA expression phenotyping is performed using EST frequency. In another aspect of the invention, RNA expression phenotyping is performed by hybridizing a sample to a high density microarray and analyzing the hybridization to one or more genes (such as those listed in Tables 1, 2, 3, 4, 5, 6 and/or 7) that are differentially expressed in samples from patients with osteoarthritis, mild osteoarthritis and/or severe ostearthritis. These gene sets have multifold uses including, but not limited to, the following examples. The expression gene sets may be used as a prognostic tool for osteoarthritis patients, to make possible more finely tuned diagnosis of osteoarthritis and allow healthcare professionals to tailor treatment to individual patients' needs. The invention can also assess the efficacy of osteoarthritis treatment by determining progression or regression of osteoarthritis in patients before, during, and after osteoarthritis treatment. Another utility of the expression gene set is in the biotechnology and pharmaceutical industries' research on disease pathway discovery for therapeutic targeting. The invention can identify alterations in gene expression in osteoarthritis and can also be used to uncover and test candidate pharmaceutical agents to treat osteoarthritis.

Nucleic Acid Members and Probes

In one aspect, the invention provides nucleic acid members and probes that bind specifically to a target nucleic acid sequence (e.g., present in a cartilage nucleic acid sample).

Nucleic acid members can be stably associated with a solid support to comprise an array according to the invention. The length of a nucleic acid member can range from 15 to 6000 nucleotides, 100 to 500 nucleotides, and in other embodiments, from 25 to 60 nucleotides. The nucleic acid members may be single or double stranded, and/or may be PCR fragments amplified from cDNA.

The invention also provides for nucleic acid sequences comprising a probe. In a certain embodiment, a probe is labeled, according to methods known in the art. A probe according to the invention is 15 to 5000 nucleotides, more preferably 100-500 nucleotides and most preferably 20 to 100 nucleotides in length. The probe may be single or double stranded, and may be a PCR fragment amplified from cDNA.

The nucleic acid members and probes according to the invention can be used to detect target sequences such as chondrocyte enriched or chondrocyte-specific RNA and preferably RNA whose presence and/or quantity in a sample are indicative, or diagnostic or prognostic, of a stage of osteoarthritis.

The target nucleic acid sequences to be analyzed are preferably from human cartilage, blood or synovial fluid and preferably comprise RNA or nucleic acid corresponding to RNA, (i.e., cDNA or amplified products of RNA or cDNAs).

Data Acquisition and Analysis of EST Sequences

The invention provides for EST sequences including “novel sequences”, “novel expressed sequence tags (ESTs)” and “known sequences” including “known sequences with a function” and “known sequences with no known function”.

The generated EST sequences are searched against available databases, including the “nt”, “nr”, “est”, “gss” and “htg” databases available through NCBI to determine putative identities for ESTs matching to known genes or other ESTs. Relative EST frequency level can then be calculated using known methods. Functional characterization of ESTs with known gene matches are made according to any known method. Preferably, generated EST sequences are compared to the non-redundant Genbank/EMBL/DDBJ and dbEST databases using the BLAST algorithm (8). A minimum value of P=10⁻¹⁰ and nucleotide sequence identity >95%, where the sequence identity is non-contiguous or scattered, are required for assignments of putative identities for ESTs matching to known genes or to other ESTs. Construction of a non-redundant list of genes represented in the EST set is done with the help of Unigene, Entrez and PubMed at the National Center for Biotechnology Information (NCBI) site (http://www.ncbi.nlm.nih.gov/). Relative gene expression frequency is calculated by dividing the number of EST copies for each gene by the total number of ESTs analyzed.

Genes are identified from ESTs according to known methods. To identify novel genes from an EST sequence, the EST should preferably be at least 100 nucleotides in length, and more preferably 150 nucleotides in length, for annotation. Preferably, the EST exhibits open reading frame characteristics (i.e., can encode a putative polypeptide).

Because of the completion of the Human Genome Project, a specific EST which matches with a genomic sequence can be mapped onto a specific chromosome based on the chromosomal location of the genomic sequence. However, no function may be known for the protein encoded by the sequence and the EST would then be considered “novel” in a functional sense. In one aspect, the invention is used to identify a novel EST which is part of a larger known sequence for which no function is known is used to determine the function of a gene comprising the EST (e.g., such as the role of expression products produced by the gene in chondrogenesis and/or in a pathology affecting chondrocytes). Alternatively, or additionally, the EST can be used to identify an mRNA or polypeptide encoded by the larger sequence as a diagnostic or prognostic marker of chondrogenesis and/or of a pathology affecting chondrocytes.

Having identified an EST corresponding to a larger sequence as chondrocyte enriched or chondrocyte-specific, other portions of the larger sequence which comprises the EST can be used in assays to elucidate gene function, e.g., to isolate polypeptides encoded by the gene, to generate antibodies specifically reactive with these polypeptides, to identify binding partners of the polypeptides (receptors, ligands, agonists, antagonists and the like) and/or to detect the expression of the gene (or lack thereof) in chondrocytes in normal, and/or diseased individuals.

In another aspect, the invention provides for nucleic acid sequences that do not demonstrate a “significant match” to any of the publicly known sequences in sequence databases at the time a query is done. Longer genomic segments comprising these types of novel EST sequences can be identified by probing genomic libraries, while longer expressed sequences can be identified in cDNA libraries and/or by performing polymerase extension reactions (e.g., RACE) using EST sequences to derive primer sequences as is known in the art. Longer fragments can be mapped to particular chromosomes by FISH and other techniques and their sequences compared to known sequences in genomic and/or expressed sequence databases and further functional analysis can be performed as described above.

Using the methods according to the invention, thousands of ESTs from the four cDNA libraries, were matched was 5,687 gene sequences, as shown in Table 1. Further, the frequency of the ESTs found in each of the four cDNA libraries that match to each gene (significant match >65%, and preferably 90% or greater, identity) was determined and those frequencies are also listed in Table 1, as well as the map location of each of the genes. Relative EST frequency is calculated by dividing the number of EST copies for each gene by the total number of ESTs analyzed. The chondrocyte-specific expression of a number of novel ESTs has been confirmed by methods known in the art. Useful methods for measuring gene expression in a tissue include RT PCR, Northern blot, etc.

Alternative methods for analyzing ESTs are also available. For example, the ESTs from each library may be assembled into contigs with sequence alignment, editing, and assembly programs such as PHRED and PHRAP (Ewing, et al., 1998, Genome Res. 3:175, incorporated herein; http://bozeman.genome.washington.edu/). Contig redundancy is reduced by clustering nonoverlapping sequence contigs using the EST clone identification number, which is common for the nonoverlapping 5 and 3 sequence reads for a single EST cDNA clone. In one aspect, the consensus sequence from each cluster is compared to the non-redundant Genbank/EMBL/DDBJ and dbEST databases using the BLAST algorithm with the help of unigene, Entrez and PubMed at the NCBI site.

In one aspect, the invention also provides for known nucleic acid sequences that are chondrocyte enriched or chondrocyte-specific.

The invention provides for known and novel nucleic acid sequences that are uniquely expressed in normal, mild osteoarthritic, and severe osteoarthritic cartilage. Table 1 also show unique known genes and names of the novel sequences identified to date in the normal, mild osteoarthritic and severe osteoarthritic cDNA libraries using the methods according to the invention.

The invention also provides for known and novel nucleic acid sequences that are up regulated and down regulated in normal, mild osteoarthritic, and severe osteoarthritic cartilage. In one aspect, nucleic acid sequences are enriched in chondrocytes compared to in chondrocytes from individuals with osteoarthritis compared to normal individuals, or in chondrocytes from particular stages of development or disease compared to particular other stages of development or disease.

The invention also provides for nucleic acid sequences that are differentially expressed in cartilage from any two of the following developmental and disease stages: normal, mild osteoarthritic and severe osteoarthritic.

### Microarrays

Nucleic acid Microarrays

Any combination of the nucleic acid members which specifically hybridize to the RNA products of the biomarkers of the invention as disclosed in Table 1 can be used for the construction of a microarray. A microarray according to the invention preferably comprises between 10 and 20,000 nucleic acid members, and more preferably comprises at least 25 nucleic acid members, but can be comprised of 50, 100, 500, 1000, 1500, 2000, 2500, 3000, 3500 or more nucleic acid members. The nucleic acid members are complementary to and specifically hybridize to the nucleic acid sequences corresponding to the biomarkers of the invention as described herein, or any combination thereof. A microarray according to the invention is used to confirm differential gene expression profiles of genes that are specifically expressed at different osteoarthritis disease stages.

The invention also provides for a microarray comprising members which specifically hybridize to the RNA products of the biomarkers disclosed which are differentially expressed between normal and osteoarthritis patients. The invention also provides for a microarray comprising members which specifically hybridize to the RNA products of the mild stage specific biomarkers. The invention also provides for a microarray comprising members which specifically hybridize to the RNA products of the severe stage specific biomarkers. Such arrays also may be used for to monitor a patient's response to therapy. Preferably, an array for osteoarthritis diagnosis comprises 10-20,000 nucleic acid members and more preferably 50-15,000 nucleic acid members. In one embodiment, the above microarrays are used to identify a therapeutic agent that modulates the level of expression of at least one nucleic acid sequence that is differentially expressed in a chondrocyte derived from any of the following chondrocyte disease stages including normal, mild osteoarthritis, moderate osteoarthritis, marked osteoarthritis and severe osteoarthritis.

In addition to native nucleic acid members which specifically hybridize to the RNA products of the biomarkers derived from the genes listed in Tables 1, 2, 3, 4, 5, 6 and/or 7, the invention also includes modified nucleic acid molecules, which include additions, substitutions, and deletions of one or more nucleotides such as the allelic variants and SNPs described above. In preferred embodiments, these modified nucleic acid molecules and/or the polypeptides they encode retain at least one activity or function of the unmodified nucleic acid molecule, such as hybridization etc. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

In the invention, standard hybridization techniques of microarray technology are utilized to assess patterns of nucleic acid expression and identify nucleic acid marker expression. Microarray technology, which is also known by other names including: DNA chip technology, gene chip technology, and solid-phase nucleic acid array technology, is well known to those of ordinary skill in the art and is based on, but not limited to, obtaining an array of identified nucleic acid probes on a substrate, labeling target molecules with reporter molecules (e.g., radioactive, chemiluminescent, or fluorescent tags such as fluorescein, Cye3-dUTP, or Cye5-dUTP), hybridizing target nucleic acids to the probes, and evaluating target-probe hybridization. A probe with a nucleic acid sequence that perfectly matches the target sequence will, in general, result in detection of a stronger reporter-molecule signal than will probes with less perfect matches. Many components and techniques utilized in nucleic acid microarray technology are presented in The Chipping Forecast, Nature Genetics, Vol. 21, January 1999, the entire contents of which is incorporated by reference herein.

According to the present invention, microarray substrates may include but are not limited to glass, silica, aluminosilicates, borosilicates, metal oxides such as alumina and nickel oxide, various clays, nitrocellulose, or nylon. According to the invention, probes are selected from the group of nucleic acids including, but not limited to: DNA, genomic DNA, cDNA, and oligonucleotides; and may be natural or synthetic. Oligonucleotide probes preferably are 20 to 25-mer oligonucleotides and DNA/cDNA probes preferably are 100 to 5000 bases in length, although other lengths may be used. Appropriate probe length may be determined by one of ordinary skill in the art by following art-known procedures. In one embodiment, preferred probes are sets of two or more of the nucleic acid molecules derived from the genes and gene sets listed in Tables 1, 2, 3, 4, 5, 6 and/or 7. Probes may be purified to remove contaminants using standard methods known to those of ordinary skill in the art such as gel filtration or precipitation.

In one embodiment, the microarray substrate may be coated with a compound to enhance synthesis of the probe on the substrate. Such compounds include, but are not limited to, oligoethylene glycols. In another embodiment, coupling agents or groups on the substrate can be used to covalently link the first nucleotide or olignucleotide to the substrate. These agents or groups may include, but are not limited to: amino, hydroxy, bromo, and carboxy groups. These reactive groups are preferably attached to the substrate through a hydrocarbyl radical such as an alkylene or phenylene divalent radical, one valence position occupied by the chain bonding and the remaining attached to the reactive groups. These hydrocarbyl groups may contain up to about ten carbon atoms, preferably up to about six carbon atoms. Alkylene radicals are usually preferred containing two to four carbon atoms in the principal chain. These and additional details of the process are disclosed, for example, in U.S. Pat. No. 4,458,066, which is incorporated by reference in its entirety.

In one embodiment, probes are synthesized directly on the substrate in a predetermined grid pattern using methods such as light-directed chemical synthesis, photochemical deprotection, or delivery of nucleotide precursors to the substrate and subsequent probe production.

In another embodiment, the substrate may be coated with a compound to enhance binding of the probe to the substrate. Such compounds include, but are not limited to: polylysine, amino silanes, amino-reactive silanes (Chipping Forecast, 1999) or chromium (Gwynne and Page, 2000). In this embodiment, presynthesized probes are applied to the substrate in a precise, predetermined volume and grid pattern, utilizing a computer-controlled robot to apply probe to the substrate in a contact-printing manner or in a non-contact manner such as ink jet or piezo-electric delivery. Probes may be covalently linked to the substrate with methods that include, but are not limited to, UV-irradiation. In another embodiment probes are linked to the substrate with heat.

Targets are nucleic acids selected from the group, including but not limited to: DNA, genomic DNA, cDNA, RNA, mRNA and may be natural or synthetic. In all embodiments, nucleic acid molecules from human cartilage, blood and synovial fluid are preferred. The tissue may be obtained from a subject or may be grown in culture (e.g. from a chondrocyte cell line). The target nucleic acid samples that are hybridized to and analyzed with a microarray of the invention are preferably from human cartilage, blood or synovial fluid and can be total RNA, mRNA or fractions thereof. A limitation for this procedure lies in the amount of RNA available for use as a target nucleic acid sample. Preferably, at least 1 microgram of total RNA is obtained for use according to this invention. This is advantageous because the amount of RNA in synovial fluid and in many cartilage biopsy samples is very minimal.

In embodiments of the invention one or more control nucleic acid molecules are attached to the substrate. Preferably, control nucleic acid molecules allow determination of factors including but not limited to: nucleic acid quality and binding characteristics; reagent quality and effectiveness; hybridization success; and analysis thresholds and success. Control nucleic acids may include but are not limited to expression products of genes such as housekeeping genes or fragments thereof.

In another embodiment, novel pharmacological agents with the potential to be useful in the treatment of osteoarthritis (OA) can be identified by assessing variations in the expression of sets of two or more OA associated nucleic acid biomarkers, corresponding to or derived from among the genes listed in Tables 1-7, prior to and after contacting chondrocyte cells or cartilage tissues with candidate pharmacological agents for the treatment of OA. The cells may be grown in culture (e.g. from a chondrocyte cell line), or may be obtained from a subject, (e.g. in a clinical trial of candidate pharmaceutical agents to treat OA). Alterations in expression of two or more sets of OA nucleic acid biomarkers, that correlate to or are derived from among the genes listed in Tables 1-7, in OA chondrocyte cells or tissues tested before and after contact with a candidate pharmacological agent to treat OA, indicate progression, regression, or stasis of OA thereby indicating efficacy of candidate agents and concomitant identification of lead compounds for therapeutic use in OA.

The invention further provides efficient methods of identifying pharmacological agents or lead compounds for agents active at the level of chondrocyte cellular function. Generally, the screening methods involve assaying for compounds that beneficially alter the OA associated nucleic acid molecule expression. Such methods are adaptable to automated, high-throughput screening of compounds.

Construction of a Microarray

In one aspect, cDNAs generated from human cartilage cDNA libraries are arrayed on a microarray. Preferably, a microarray according to the invention comprises chondrocyte enriched or chondrocyte-specific genes and includes the whole spectrum of genes that are important in the osteoarthritis disease process. In another aspect, ESTs corresponding to the biomarkers of the invention are arrayed onto a microarray.

The EST frequency analysis in Tables 1-7 shows the differential gene expression profiles for known genes. Microarrays according to the invention may be used to confirm these profiles and may also be used to show differential expression profiles between different osteoarthritis disease states for diagnostic purposes.

In the subject methods, an array of nucleic acid members stably associated with the surface of a substantially solid support is contacted with a sample comprising target nucleic acids under hybridization conditions sufficient to produce a hybridization pattern of complementary nucleic acid members/target complexes in which one or more complementary nucleic acid members at unique positions on the array specifically hybridize to target nucleic acids. The identity of target nucleic acids which hybridize can be determined with reference to location of nucleic acid members on the array.

The nucleic acid members may be produced using established techniques such as polymerase chain reaction (PCR) and reverse transcription (RT). These methods are similar to those currently known in the art (see e.g., PCR Strategies, Michael A. Innis (Editor), et al. (1995) and PCR: Introduction to Biotechniques Series, C. R. Newton, A. Graham (1997)). Amplified nucleic acids are purified by methods well known in the art (e.g., column purification or alcohol precipitation). A nucleic acid is considered pure when it has been isolated so as to be substantially free of primers and incomplete products produced during the synthesis of the desired nucleic acid. Preferably, a purified nucleic acid will also be substantially free of contaminants which may hinder or otherwise mask the specific binding activity of the molecule.

A microarray according to the invention comprises a plurality of unique nucleic acids attached to one surface of a solid support at a density exceeding 20 different nucleic acids/cm², where each of the nucleic acids is attached to the surface of the solid support in a non-identical pre-selected region. Each associated sample on the array comprises a nucleic acid composition, of known identity, usually of known sequence, as described in greater detail below. Any conceivable substrate may be employed in the invention.

In one embodiment, the nucleic acid attached to the surface of the solid support is DNA. In a preferred embodiment, the nucleic acid attached to the surface of the solid support is cDNA or RNA. In another preferred embodiment, the nucleic acid attached to the surface of the solid support is cDNA synthesized by polymerase chain reaction (PCR). Preferably, a nucleic acid member in the array, according to the invention, is at least 50 nucleotides in length. In one embodiment, a nucleic acid member is at least 150 nucleotides in length. Preferably, a nucleic acid member is less than 1000 nucleotides in length. More preferably, a nucleic acid member is less than 500 nucleotides in length. In one embodiment, an array comprises at least 10 different nucleic acids attached to one surface of the solid support. In another embodiment, the array comprises at least 100 different nucleic acids attached to one surface of the solid support. In yet another embodiment, the array comprises at least 10,000 different nucleic acids attached to one surface of the solid support. In yet another embodiment, the array comprises at least 15,000 different nucleic acids attached to one surface of the solid support.

In the arrays of the invention, the nucleic acid compositions are stably associated with the surface of a solid support, where the support may be a flexible or rigid solid support. By “stably associated” is meant that each nucleic acid member maintains a unique position relative to the solid support under hybridization and washing conditions. As such, the samples are non-covalently or covalently stably associated with the support surface. Examples of non-covalent association include non-specific adsorption, binding based on electrostatic interactions (e.g., ion pair interactions), hydrophobic interactions, hydrogen bonding interactions, specific binding through a specific binding pair member covalently attached to the support surface, and the like. Examples of covalent binding include covalent bonds formed between the nucleic acids and a functional group present on the surface of the rigid support (e.g., —OH), where the functional group may be naturally occurring or present as a member of an introduced linking group, as described in greater detail below The amount of nucleic acid present in each composition will be sufficient to provide for adequate hybridization and detection of target nucleic acid sequences during the assay in which the array is employed. Generally, the amount of each nucleic acid member stably associated with the solid support of the array is at least about 0.001 ng, preferably at least about 0.02 ng and more preferably at least about 0.05 ng, where the amount may be as high as 1000 ng or higher, but will usually not exceed about 20 ng. Where the nucleic acid member is “spotted” onto the solid support in a spot comprising an overall circular dimension, the diameter of the “spot” will generally range from about 10 to 5,000 μm, usually from about 20 to 2,000 μm and more usually from about 100 to 200 μm.

Control nucleic acid members may be present on the array including nucleic acid members comprising oligonucleotides or nucleic acids corresponding to genomic DNA, housekeeping genes, vector sequences, plant nucleic acid sequence, negative and positive control genes, and the like. Control nucleic acid members are calibrating or control genes whose function is not to tell whether a particular “key” gene of interest is expressed, but rather to provide other useful information, such as background or basal level of expression.

Other control nucleic acids are spotted on the array and used as target expression control nucleic acids and mismatch control nucleotides to monitor non-specific binding or cross-hybridization to a nucleic acid in the sample other than the target to which the probe is directed. Mismatch probes thus indicate whether a hybridization is specific or not. For example, if the target is present, the perfectly matched probes should be consistently brighter than the mismatched probes. In addition, if all control mismatches are present, the mismatch probes are used to detect a mutation.

Solid Surface

An array according to the invention can comprise either a flexible or rigid substrate, and has a surface which allows attachment of nucleic acid members. A flexible substrate is capable of being bent, folded or similarly manipulated without breakage. Examples of solid materials which are flexible solid supports with respect to the present invention include membranes, e.g., nylon, flexible plastic films, and the like. By “rigid” is meant that the support is solid and does not readily bend, i.e., the support is not flexible. As such, the rigid substrates of the subject arrays are sufficient to provide physical support and structure to the associated nucleic acids present thereon under the assay conditions in which the array is employed, particularly under high throughput handling conditions. In some embodiments, an array can consist of surfaces for attachment of nucleic acid members which are arranged in three dimensions, for example a tube which allows greater hybridization interaction.

The substrate may be biological, non-biological, organic, inorganic, or a combination of any of these, existing as particles, strands, precipitates, gels, sheets, tubing, spheres, beads, containers, capillaries, pads, slices, films, plates, slides, chips, etc. The substrate may have any convenient shape, such as a disc, square, sphere, circle, etc. The substrate is preferably flat or planar but may take on a variety of alternative surface configurations. The substrate may be a polymerized Langmuir Blodgett film, functionalized glass, Si, Ge, GaAs, GaP, SiO₂, SIN₄, modified silicon, or any one of a wide variety of gels or polymers such as (poly)tetrafluoroethylene, (poly)vinylidenedifluoride, polystyrene, polycarbonate, or combinations thereof. Other substrate materials will be readily apparent to those of skill in the art upon review of this disclosure.

In a preferred embodiment the substrate is flat glass or single-crystal silicon. According to some embodiments, the surface of the substrate is etched using well-known techniques to provide for desired surface features. For example, by way of formation of trenches, v-grooves, mesa structures, or the like, the synthesis regions may be more closely placed within the focus point of impinging light, be provided with reflective “mirror” structures for maximization of light collection from fluorescent sources, etc.

Surfaces on the solid substrate will usually, though not always, be composed of the same material as the substrate. Alternatively, the surface may be composed of any of a wide variety of materials, for example, polymers, plastics, resins, polysaccharides, silica or silica-based materials, carbon, metals, inorganic glasses, membranes, or any of the above-listed substrate materials. In some embodiments the surface may provide for the use of caged binding members which are attached firmly to the surface of the substrate. Preferably, the surface will contain reactive groups, which are carboxyl, amino, hydroxyl, or the like. Most preferably, the surface will be optically transparent and will have surface Si—OH functionalities, such as are found on silica surfaces.

The surface of the substrate is preferably provided with a layer of linker molecules, although it will be understood that the linker molecules are not required elements of the invention. The linker molecules are preferably of sufficient length to permit nucleic acids of the invention and on a substrate to hybridize to other nucleic acid molecules and to interact freely with molecules exposed to the substrate.

Often, the substrate is a silicon or glass surface, (poly)tetrafluoroethylene, (poly)vinylidendifluoride, polystyrene, polycarbonate, a charged membrane, such as nylon 66 or nitrocellulose, or combinations thereof. In a preferred embodiment, the solid support is glass. Preferably, at least one surface of the substrate will be substantially flat. Preferably, the surface of the solid support will contain reactive groups, including, but not limited to, carboxyl, amino, hydroxyl, thiol, or the like. In one embodiment, the surface is optically transparent. In a preferred embodiment, the substrate is a poly-lysine coated slide or Gamma amino propyl silane-coated Corning Microarray Technology-GAPS or CMT-GAP2 coated slides.

An y solid support to which a nucleic acid member may be attached may be used in the invention. Examples of suitable solid support materials include, but are not limited to, silicates such as glass and silica gel, cellulose and nitrocellulose papers, nylon, polystyrene, polymethacrylate, latex, rubber, and fluorocarbon resins such as TEFLON™.

The solid support material may be used in a wide variety of shapes including, but not limited to slides and beads. Slides provide several functional advantages and thus are a preferred form of solid support. Due to their flat surface, probe and hybridization reagents are minimized using glass slides. Slides also enable the targeted application of reagents, are easy to keep at a constant temperature, are easy to wash and facilitate the direct visualization of RNA and/or DNA immobilized on the solid support. Removal of RNA and/or DNA immobilized on the solid support is also facilitated using slides.

The particular material selected as the solid support is not essential to the invention, as long as it provides the described function. Normally, those who make or use the invention will select the best commercially available material based upon the economics of cost and availability, the expected application requirements of the final product, and the demands of the overall manufacturing process.

Spotting Method

In one aspect, the invention provides for arrays where each nucleic acid member comprising the array is spotted onto a solid support.

Preferably, spotting is carried out as follows. PCR products (˜40μ) of cDNA clones from osteoarthritis, or normal cartilage cDNA libraries, in the same 96-well tubes used for amplification, are precipitated with 41 μl ( 1/10 volume) of 3M sodium acetate (pH 5.2) and 100 μl (2.5 volumes) of ethanol and stored overnight at −20° C. They are then centrifuged at 3,300 rpm at 4° C. for 1 hour. The obtained pellets are washed with 50 μlice-cold 70% ethanol and centrifuged again for 30 minutes. The pellets are then air-dried and resuspended well in 20 ul 3×SSC or in 50% dimethylsulfoxide (DMSO) overnight. The samples are then spotted, either singly or in duplicate, onto polylysine-coated slides (Sigma Cat. No. P0425) using a robotic GMS 417 or 427 arrayer (Affymetrix, Ca).

The boundaries of the spots on the microarray may be marked with a diamond scriber (as the spots become invisible after post-processing). The arrays are rehydrated by suspending the slides over a dish of warm particle free ddH₂O for approximately one minute (the spots will swell slightly but will not run into each other) and snap-dried on a 70-80° C. inverted heating block for 3 seconds. Nucleic acid is then UV crosslinked to the slide (Stratagene, Stratalinker, 65 mJ—set display to “650” which is 650×100 μJ) or the array is baked at 80 C for two to four hours prior to hybridization. The arrays are placed in a slide rack. An empty slide chamber is prepared and filled with the following solution: 3.0 grams of succinic anhydride (Aldrich) was dissolved in 189 ml of 1-methyl-2-pyrrolidinone (rapid addition of reagent is crucial); immediately after the last flake of succinic anhydride is dissolved, −21.0 ml of 0.2 M sodium borate is mixed in and the solution is poured into the slide chamber. The slide rack is plunged rapidly and evenly in the slide chamber and vigorously shaken up and down for a few seconds, making sure the slides never leave the solution, and then mixed on an orbital shaker for 15-20 minutes. The slide rack is then gently plunged in 95° C. ddH₂O for 2 minutes, followed by plunging five times in 95% ethanol. The slides are then air dried by allowing excess ethanol to drip onto paper towels. The arrays are stored in the slide box at room temperature until use.

Numerous methods may be used for attachment of the nucleic acid members of the invention to the substrate (a process referred to as “spotting”). For example, nucleic acids are attached using the techniques of, for example U.S. Pat. No. 5,807,522, which is incorporated herein by reference, for teaching methods of polymer attachment.

Alternatively, spotting may be carried out using contact printing technology as is known in the art.

Screening Methods

The present invention also provides methods for identifying modulators which bind to the RNA products of OA biomarkers, in particular the OA biomarkers listed in Tables 1-7, or have a modulatory effect on the expression or activity of RNA products of one or more OA biomarkers. Modulators which decrease the expression or activity of one or more OA biomarker gene products which are upregulated in OA disease state relative to a normal state are believed to be useful in treating OA. Conversely modulators which increase the expression or activity of one or more OA biomarkers which are downregulated in an OA disease state relative to a normal state are also believed to be useful in treating OA. Such screening assays are known to those of skill in the art and include, without limitation, cell-based assays and cell free assays.

Molecules identified in screening assays as being capable of modulating expression of the OA biomarkers identified herein are key candidates for further evaluation for use in the treatment of OA. In a preferred embodiment, these molecules will downregulate expression and/or activity of products of those OA biomarkers that have increased expression in the OA diseased state relative to a normal state, and/or upregulate expression and/or activity of those OA biomarkers that have decreased expression in the OA diseased state relative to normal state.

Screening assay mixtures comprise a candidate pharmacological agent. Typically, a plurality of assay mixtures are run in parallel with different agent concentrations to obtain a different response to the various concentrations. Typically, one of these concentrations serves as a negative control, i.e., at zero concentration of agent or at a concentration of agent below the limits of assay detection. Candidate agents encompass numerous chemical classes, although typically they are organic compounds. Preferably, the candidate pharmacological agents are small organic compounds, i.e., those having a molecular weight of more than 50 yet less than about 2500, preferably less than about 1000 and, more preferably, less than about 500. Candidate agents comprise functional chemical groups necessary for structural interactions with nucleic acids, and typically include at least an amine, carbonyl, hydroxyl, or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid, the agent typically is a DNA or RNA molecule, although modified nucleic acids as defined herein are also contemplated.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant, and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease, inhibitors, nuclease inhibitors, antimicrobial agents, and the like may also be used.

The order of addition of components, incubation temperature, time of incubation, and other parameters of the assay may be readily determined. Such experimentation merely involves optimization of the assay parameters, not the fundamental composition of the assay. Incubation temperatures typically are between 4 and 40 C. Incubation times preferably are minimized to facilitate rapid, high throughput screening, and typically are between 0.1 and 10 hours.

The mixture of the foregoing assay materials is incubated under conditions whereby, (a) the anti-OA candidate agent is incubated with a cell expressing RNA products of one or more biomarkers of the invention; (b) the amount of the RNA products present in (a) is determined; and (c) the amount in (a) is compared to that present in a corresponding control cell that has not been contacted with the test compound, so that if the amount of RNA product is altered relative to the amount in the control, a compound to be tested for an ability to prevent, treat, manage or ameliorate osteoarthritis or a symptom thereof is identified. In a specific embodiment, the expression level(s) is altered by 5%, 10%, 15%, 25%, 30%, 40%, 50%, 5 to 25%, 10 to 30%, at least 1 fold, at least 1.5 fold, at least 2 fold, 4 fold, 5 fold, 10 fold, 25 fold, 1 to 10 fold, or 5 to 25 fold relative to the expression level in the control as determined by utilizing an assay described herein (e.g., a microarray or RT-PCR) or an assay well known to one of skill in the art. In alternate embodiments, such a method comprises determining the amount of RNA product of at least 2, at least 3, at least 4, at least 5, at least 6, at least 7, at least 8, at least 9, at least 10, at least 12, at least 15, at least 20, at least 25, at least 30, at least 35, at least 40, at least 45, at least 50, at least 100, at least 200 or more 1 to 5, 1-10, 5-10, 5-25, or 10-40, all or any combination of the biomarkers of the invention present in the cell and comparing the amounts to those present in the control.

The cells utilized in the cell-based assays described herein can be engineered to express a biomarker of the invention utilizing techniques known in the art. See, e.g., Section III entitled “Recombinant Expression Vectors and Host Cells” of U.S. Pat. No. 6,245,527, which is incorporated herein by reference. Alternatively, cells that endogenously express a biomarker of the invention can be used. For example, chondrocyte cells may be used.

In a specific embodiment, chondrocytes are isolated from a “normal” individual, or an individual with mild, moderate, marked or severe osteoarthritis and are incubated in the presence and absence of a test compound for varying amounts of time (i.e., 30 min, 1 hr, 5 hr, 24 hr, 48 hr and 96 hrs). When screening for prophylactic or therapeutic agents, a clone of the full sequence of a biomarker of the invention or functional portion thereof is used to transfect chondrocytes. The transfected chondrocytes are cultured for varying amounts of time (i.e., 1, 2, 3, 5, 7, 10, or 14 days) in the presence or absence of test compound. Following incubation, target nucleic acid samples are prepared from the chondrocytes and hybridized to a nucleic acid probe corresponding to a nucleic acid sequence which is differentially expressed in a chondrocyte derived from at least any two of the following of: normal, mild osteoarthritic, moderate osteoarthritic and severe osteoarthritic. The nucleic acid probe is labeled, for example, with a radioactive label, according to methods well-known in the art and described herein. Hybridization is carried out by northern blot, for example as described in Ausubel et al., supra or Sambrook et al., supra). The differential hybridization, as defined herein, of the target to the samples on the array from normal relative to RNA from any one of mild osteoarthritic, moderate osteoarthritic, marked osteoarthritic and severe osteoarthritic is indicative of the level of expression of RNA corresponding to a differentially expressed chondrocyte specific nucleic acid sequence. A change in the level of expression of the target sequence as a result of the incubation step in the presence of the test compound, is indicative of a compound that increases or decreases the expression of the corresponding chondrocyte specific nucleic acid sequence.

The present invention also provides a method for identifying a compound to be tested for an ability to prevent, treat, manage or ameliorate osteoarthritis or a symptom thereof, said method comprises: (a) contacting a cell-free extract (e.g., a chondrocyte extract) with a nucleic acid sequence encoding the RNA product of one or more biomarkers of the invention and a test compound; (b) determining the amount of RNA product present in (a); and (c) comparing the amount(s) in (a) to that present to a corresponding control that has not been contacted with the test compound, so that if the amount of the RNA product is altered relative to the amount in the control, a compound to be tested for an ability to prevent, treat, manage or ameliorate osteoarthritis or a symptom thereof is identified. In a specific embodiment, the expression level(s) is altered by 5%, 10%, 15%, 25%, 30%, 40%, 50%, 5 to 25%, 10 to 30%, at least 1 fold, at least 1.5 fold, at least 2 fold, 4 fold, 5 fold, 10 fold, 25 fold, 1 to 10 fold, or 5 to 25 fold relative to the expression level in the control sample determined by utilizing an assay described herein (e.g., a microarray or RT-PCR) or an assay well known to one of skill in the art. In alternate embodiments, such a method comprises determining the amount of RNA product of at least 2, at least 3, at least 4, at least 5, at least 6, at least 7, at least 8, at least 9, at least 10, at least 12, at least 15, at least 20, at least 25, at least 30, at least 35, at least 40, at least 45, at least 50, 1 to 5, 1-10, 5-10, 5-25, or 10-40, all or any combination of the biomarkers of the invention present in the extract and comparing the amounts to those present in the control.

The invention also provides a screening method for OA gene-specific binding agents. For example, OA gene-specific pharmacological agents identified using such a screen are useful in a variety of diagnostic and therapeutic applications as described herein. In general, the specificity of an OA associated gene binding to a binding agent is shown by binding equilibrium constants. Targets that are capable of selectively binding an OA associated gene preferably have binding equilibrium constants of at least about 10E7 M.per1, more preferably at least about 10.E8 M.per 1, and most preferably at least about 10.E9 M. per 1. The wide variety of cell-based and cell-free assays may be used to demonstrate osteoarthritis gene-specific binding. Cell-based assays include one, two and three hybrid screens, assays in which osteoarthritic gene-mediated transcription is inhibited or increased, etc. Cell-free assays include osteoarthritic gene-protein binding assays, immunoassays, etc. Other assays useful for screening agents which bind osteoarthritic polypeptides include fluorescence resonance energy transfer (FRET), and electrophoretic mobility shift analysis (EMSA).

Accepted animal models can be utilized to determine the efficacy of the compounds identified via the in vitro screening methods described above for the prevention, treatment, management and/or amelioration of osteoarthritis or a symptom thereof. Such models can include the various experimental animal models of inflammatory arthritis known in the art and described in Crofford L. J. and Wilder R. L., “Arthritis and Autoimmunity in Animals”, in Arthritis and Allied Conditions: A Textbook of Rheumatology, McCarty et al. (eds.), Chapter 30 (Lee and Febiger, 1993). The principle animal models for arthritis or inflammatory disease known in the art and widely used include: adjuvant-induced arthritis rat models, collagen-induced arthritis rat and mouse models and antigen-induced arthritis rat, rabbit and hamster models, all described in Crofford L. J. and Wilder R. L., “Arthritis and Autoimmunity in Animals”, in Arthritis and Allied Conditions: A Textbook of Rheumatology, McCarty et al. (eds.), Chapter 30 (Lee and Febiger, 1993), incorporated herein by reference in its entirety.

In one embodiment, the efficacy of a compound for the prevention, treatment, management and/or amelioration of osteoarthritis or a symptom thereof is determined using a carrageenan-induced arthritis rat model. Carrageenan-induced arthritis has also been used in rabbit, dog and pig in studies of chronic arthritis or inflammation. Quantitative histomorphometric assessment is used to determine therapeutic efficacy. The methods for using such a carrageenan-induced arthritis model is described in Hansra P. et al., “Carrageenan-Induced Arthritis in the Rat,” Inflammation, 24(2): 141-155, (2000). Also commonly used are zymosan-induced inflammation animal models as known and described in the art.

The anti-inflammatory activity of the compounds can be assessed by measuring the inhibition of carrageenan-induced paw edema in the rat, using a modification of the method described in Winter C. A. et al., “Carrageenan-Induced Edema in Hind Paw of the Rat as an Assay for Anti-inflammatory Drugs” Proc. Soc. Exp. Biol Med. 111, 544-547, (1962). This assay has been used as a primary in vivo screen for the anti-inflammatory activity of most NSAIDs, and is considered predictive of human efficacy. The anti-inflammatory activity of the test compound is expressed as the percent inhibition of the increase in hind paw weight of the test group relative to the vehicle dosed control group.

In another embodiment, the efficacy of a compound for the prevention, treatment, management and/or amelioration of osteoarthritis or a symptom thereof is determined using a collagen-induced arthritis (CIA) model. CIA is an animal model for the human autoimmune disease rheumatoid arthritis (RA) (Trenthom et al., 1977, J. Exp. Med., 146:857). This disease can be induced in many species by the administration of heterologous type II collagen (Courtenay et al., 1980, Nature 283:665; Cathcart et at, 1986, Lab. Invest., 54:26). With respect to animal models of arthritis see, in addition, e.g., Holmdahl, R., 1999, Curr. Biol. 15:R528-530.

In another embodiment, the efficacy of a compound for the prevention, treatment, management and/or amelioration of osteoarthritis or a symptom thereof is determined using assays that determine bone formation and/or bone loss. Animal models such as ovariectomy-induced bone resorption mice, rat and rabbit models are known in the art for obtaining dynamic parameters for bone formation. Using methods such as those described by Yositake et al. or Yamamoto et al., bone volume is measured in vivo by microcomputed tomography analysis and bone histomorphometry analysis. Yoshitake et al., “Osteopontin-Deficient Mice Are Resist ant to Ovariectomy-Induced Bone Resorption,” Proc. Natl. Acad. Sci. 96:8156-8160, (1999); Yamamoto et al., “The Integrin Ligand Echistatin Prevents Bone Loss in Ovariectomized Mice and Rats,” Endocrinology 139(3):1411-1419, (1998), both incorporated herein by reference in their entirety.

In another aspect of the invention, pre- and post-treatment alterations in expression of two or more of the osteoarthritic nucleic acid markers including, but not limited to, biomarkers derived from genes listed in Tables 1, 2, 3, 4, 5, 6, and/or 7 in cartilage, blood and/or synovial cells or tissues from individuals with osteoarthritis may be used to assess treatment parameters including, but not limited to: dosage, method of administration, timing of administration, and combination with other treatments as described herein.

Kits

The invention provides for kits for performing expression assays for both diagnostic and screening purposes using the biomarkers of the present invention. Such kits according to the subject invention include those having an array of the invention having associated nucleic acid members and packaging means therefore. In another embodiment, kits of the invention may comprise reagents employed to detect and quantitate gene expression of the biomarkers of the invention and can include such things as: 1) primers for generating test nucleic acids; 2) dNTPs and/or rNTPs (either premixed or separate), optionally with one or more uniquely labeled dNTPs and/or rNTPs (e.g., biotinylated or Cy3 or Cy5 tagged dNTPs); 3) post synthesis labeling reagents, such as chemically active derivatives of fluorescent dyes; 4) enzymes, such as reverse transcriptases, DNA polymerases, and the like; 5) various buffer mediums, e.g., hybridization and washing buffers; 6) labeled probe purification reagents and components, like spin columns, etc.; and 7) signal generation and detection reagents, e.g., streptavidin-alkaline phosphatase conjugate, hemifluorescent or chemiluminescent substrate, and the like.

Use of a Microarray

Nucleic acid arrays according to the invention can be used in high throughput techniques that can assay a large number of nucleic acids in a sample comprising one or more target nucleic acid sequences. The arrays of the subject invention find use in a variety of applications, including gene expression analysis, diagnosis of osteoarthritis and prognosis of osteoarthritis as well as monitoring a patient's response to therapy, drug screening, and the like.

In one aspect, the arrays of the invention are used in, among other applications, differential gene expression assays. For example, arrays are useful in the differential expression analysis of: (a) diseased osteoarthritis and normal tissue; (b) tissues representing different stages of osteoarthritis; (c) developing cartilage (e.g., fetal cartilage??); (d) chondrocyte responses to external or internal stimuli; (e) cartilage/chondrocyte response to treatment; (f) cartilage tissue engineering; (g) pharmacogenomics; and the like. The arrays are also useful in broad scale expression screening for drug discovery and research, such as the effect of a particular active agent on the expression pattern of genes in a particular cell, where such information is used to reveal drug efficacy and toxicity, environmental monitoring, disease research and the like. For example, high expression of a particular nucleic acid sequence in an osteoarthritis sample (mild, moderate, marked, or severe), which is not observed in a corresponding normal cell, can indicate an osteoarthritis-specific gene product.

Target Preparation

The targets for the microarrays according to the invention are preferably derived from human cartilage, blood or synovial fluid.

A target nucleic acid is capable of binding to a nucleic acid probe or nucleic acid member of complementary sequence through one or more types of chemical bonds, usually through complementary base pairing, usually through hydrogen bond formation.

As used herein, a “nucleic acid derived from an mRNA transcript: or a “nucleic acid corresponding to an mRNA” refers to a nucleic acid for which synthesis of the mRNA transcript or a sub-sequence thereof has ultimately served as a template. Thus, a cDNA reverse transcribed from an mRNA, an RNA transcribed from that cDNA, a DNA amplified from the cDNA, an RNA transcribed from the amplified DNA, etc. are all derived from or correspond to the mRNA transcript and detection of such derived or corresponding products is indicative of or proportional to the presence and/or abundance of the original transcript in a sample. Thus, suitable target nucleic acid samples include, but are not limited to, mRNA transcripts of a gene or genes, cDNA reverse transcribed from the mRNA, cRNA transcribed from the cDNA, DNA amplified from a gene or genes, RNA transcribed from amplified DNA, and the like. The nucleic acid targets used herein are preferably derived from human cartilage, blood or synovial fluid. Preferably, the targets are nucleic acids derived from human cartilage, blood or synovial fluid extracts. Nucleic acids can be single- or double-stranded DNA, RNA, or DNA-RNA hybrids synthesized from human cartilage, blood or synovial fluid mRNA extracts using methods known in the art, for example, reverse transcription or PCR.

In the simplest embodiment, such a nucleic acid target comprises total mRNA or a nucleic acid sample corresponding to mRNA (e.g., cDNA) isolated from cartilage, blood, or synovial fluid samples. In another embodiment, total mRNA is isolated from a given sample using, for example, an acid guanidinium-phenol-chloroform extraction method and polyA+ mRNA is isolated by oligo dT column chromatography or by using (dT)ₙ magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987). In a preferred embodiment, total RNA is extracted using TRIzol® reagent (GIBCO/BRL, Invitrogen Life Technologies, Cat. No. 15596). Purity and integrity of RNA is assessed by absorbance at 260/280 nm and agarose gel electrophoresis followed by inspection under ultraviolet light.

In some embodiments, it is desirable to amplify the target nucleic acid sample prior to hybridization, for example, when synovial fluid is used. One of skill in the art will appreciate that whatever amplification method is used, if a quantitative result is desired, care must be taken to use a method that maintains or controls for the relative frequencies of the amplified nucleic acids. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified nucleic acid. Detailed protocols for quantitative PCR are provided in PCR Protocols, A Guide to Methods and Applications, Innis et al., Academic Press, Inc. N.Y., (1990) and (Heid et al., 1996; Morrison et al., 1998). We used real time quantitative RT-PCR to analyse the distribution of OA associated mRNA in samples.

Other suitable amplification methods include, but are not limited to polymerase chain reaction (PCR) (Innis, et al., PCR Protocols. A Guide to Methods and Application. Academic Press, Inc. San Diego, (1990)), ligase chain reaction (LCR) (see Wu and Wallace, 1989, Genomics, 4:560; Landegren, et al., 1988, Science, 241:1077 and Barringer, et al., 1990, Gene, 89:117, transcription amplification (Kwoh, et al., 1989, Proc. Natl. Acad. Sci. USA, 86: 1173), and self-sustained sequence replication (Guatelli, et al., 1990, Proc. Nat. Acad. Sci. USA, 87: 1874).

In a particularly preferred embodiment, the target nucleic acid sample mRNA is reverse transcribed with a reverse transcriptase and a primer consisting of oligo dT and a sequence encoding the phage T7 promoter to provide single-stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. Methods of in vitro transcription are well known to those of skill in the art (see, e.g., Sambrook, supra.) and this particular method is described in detail by Van Gelder, et al., 1990, Proc. Natl. Acad. Sci. USA, 87: 1663-1667 who demonstrate that in vitro amplification according to this method preserves the relative frequencies of the various RNA transcripts. Moreover, Eberwine et al. Proc. Natl. Acad. Sci. USA, 89: 3010-3014 provide a protocol that uses two rounds of amplification via in vitro transcription to achieve greater than 10⁶ fold amplification of the original starting material thereby permitting expression monitoring even where biological samples are limited.

Labeling of Target or Nucleic Acid Probe

Either the target or the probe can be labeled.

Any analytically detectable marker that is attached to or incorporated into a molecule may be used in the invention. An analytically detectable marker refers to any molecule, moiety or atom which is analytically detected and quantified.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241, the entireties of which are incorporated by reference herein.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The labels may be incorporated by any of a number of means well known to those of skill in the art. However, in a preferred embodiment, the label is simultaneously incorporated during the amplification step in the preparation of the sample nucleic acids. Thus, for example, polymerase chain reaction (PCR) with labeled primers or labeled nucleotides will provide a labeled amplification product. In a preferred embodiment, transcription amplification, as described above, using a labeled nucleotide (e.g. fluorescein-labeled UTP and/or CTP) incorporates a label into the transcribed nucleic acids.

Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example, nick translation or end-labeling (e.g. with a labeled RNA) by kinasing of the nucleic acid and subsequent attachment (ligation) of a nucleic acid linker joining the sample nucleic acid to a label (e.g., a fluorophore).

In a preferred embodiment, the fluorescent modifications are by cyanine dyes e.g. Cy-3/Cy-5 dUTP, Cy-3/Cy-5 dCTP (Amersham Pharmacia) or alexa dyes (Khan, et al., 1998, Cancer Res. 58:5009-5013).

In a preferred embodiment, the two target samples used for comparison are labeled with different fluorescent dyes which produce distinguishable detection signals, for example, targets made from normal cartilage are labeled with Cy5 and targets made from mild osteoarthritis cartilage are labeled with Cy3. The differently labeled target samples are hybridized to the same microarray simultaneously. In a preferred embodiment, the labeled targets are purified using methods known in the art, e.g., by ethanol purification or column purification.

In a preferred embodiment, the target will include one or more control molecules which hybridize to control probes on the microarray to normalize signals generated from the microarray. Preferably, labeled normalization targets are nucleic acid sequences that are perfectly complementary to control oligonucleotides that are spotted onto the microarray as described above. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes, thereby normalizing the measurements.

Preferred normalization targets are selected to reflect the average length of the other targets present in the sample, however, they are selected to cover a range of lengths. The normalization control(s) also can be selected to reflect the (average) base composition of the other probes in the array, however, in a preferred embodiment, only one or a few normalization probes are used and they are selected such that they hybridize well (i.e., have no secondary structure and do not self hybridize) and do not match any target molecules.

Normalization probes are localized at any position in the array or at multiple positions throughout the array to control for spatial variation in hybridization efficiency. In a preferred embodiment, normalization controls are located at the corners or edges of the array as well as in the middle.

Hybridization Conditions

Nucleic acid hybridization involves providing a denatured probe or target nucleic acid member and target nucleic acid under conditions where the probe or target nucleic acid member and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

The invention provides for hybridization conditions comprising the Dig hybridization mix (Boehringer); or formamide-based hybridization solutions, for example as described in Ausubel et al., supra and Sambrook et al. supra.

Methods of optimizing hybridization conditions are well known to those of skill in the art (see, e.g., Laboratory Techniques in Biochemistry and Molecular Biology, Vol. 24: Hybridization With Nucleic acid Probes, P. Tijssen, ed. Elsevier, N.Y., (1993)).

Following hybridization, non-hybridized labeled or unlabeled nucleic acid is removed from the support surface, conveniently by washing, thereby generating a pattern of hybridized target nucleic acid on the substrate surface. A variety of wash solutions are known to those of skill in the art and may be used. The resultant hybridization patterns of labeled, hybridized oligonucleotides and/or nucleic acids may be visualized or detected in a variety of ways, with the particular manner of detection being chosen based on the particular label of the test nucleic acid, where representative detection means include scintillation counting, autoradiography, fluorescence measurement, calorimetric measurement, light emission measurement and the like.

Image Acquisition and Data Analysis

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding to the signal emitted by a known number of end labeled target nucleic acids to obtain a count or absolute value of the copy number of each end-labeled target that is hybridized to a particular spot on the array in the hybridization pattern.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e., data deviating from a predetermined statistical distribution, and calculating the relative binding affinity of the test nucleic acids from the remaining data. The resulting data is displayed as an image with the intensity in each region varying according to the binding affinity between associated oligonucleotides and/or nucleic acids and the test nucleic acids.

The following detection protocol is used for the simultaneous analysis of two cartilage samples to be compared, where each sample is labeled with a different fluorescent dye.

Each element of the microarray is scanned for the first fluorescent color. The intensity of the fluorescence at each array element is proportional to the expression level of that gene in the sample.

The scanning operation is repeated for the second fluorescent label. The ratio of the two fluorescent intensities provides a highly accurate and quantitative measurement of the relative gene expression level in the two tissue samples.

In a preferred embodiment, fluorescence intensities of immobilized target nucleic acid sequences were determined from images taken with a custom confocal microscope equipped with laser excitation sources and interference filters appropriate for the Cy3 and Cy5 fluors. Separate scans were taken for each fluor at a resolution of 225 μm² per pixel and 65,536 gray levels. Image segmentation to identify areas of hybridization, normalization of the intensities between the two fluor images, and calculation of the normalized mean fluorescent values at each target are as described (Khan, et al., 1998, Cancer Res. 58:5009-5013. Chen, et al., 1997, Biomed. Optics 2:364-374). Normalization between the images is used to adjust for the different efficiencies in labeling and detection with the two different fluors. This is achieved by equilibrating to a value of one the signal intensity ratio of a set of internal control genes spotted on the array.

In another preferred embodiment, the array is scanned in the Cy 3 and Cy5 channels and stored as separate 16-bit TIFF images. The images are incorporated and analysed using software which includes a gridding process to capture the hybridization intensity data from each spot on the array. The fluorescence intensity and background-subtracted hybridization intensity of each spot is collected and a ratio of measured mean intensities of Cy5 to Cy3 is calculated. A liner regression approach is used for normalization and assumes that a scatter plot of the measured Cy5 versus Cy3 intensities should have a scope of one. The average of the ratios is calculated and used to rescale the data and adjust the slope to one. A post-normalization cutoff of greater than 1.0 fold up- or down-regulation is used to identify differentially expressed genes.

Following detection or visualization, the hybridization pattern is used to determine quantitative information about the genetic profile of the labeled target nucleic acid sample that was contacted with the array to generate the hybridization pattern, as well as the physiological source from which the labeled target nucleic acid sample was derived. By “genetic profile” is meant information regarding the types of nucleic acids present in the sample, e.g., such as the types of genes to which they are complementary, and/or the copy number of each particular nucleic acid in the sample. From this data, one can also derive information about the physiological source from which the target nucleic acid sample was derived, such as the types of genes expressed in the tissue or cell which is the physiological source of the target, as well as the levels of expression of each gene, particularly in quantitative terms.

Where one uses the subject methods to compare target nucleic acids from two or more physiological sources, the hybridization patterns may be compared to identify differences between the patterns. Where arrays in which each of the different nucleic acid members corresponds to a known gene are employed, any discrepancies are related to a differential expression of a particular gene in the physiological sources being compared. Thus, the subject methods find use in differential gene expression assays, where one may use the subject methods in the differential expression analysis of: (a) diseased vs. normal tissue, e.g., osteoarthritic and normal tissue, (b) tissue derived from different stages of osteoarthritis; and the like.

In a particularly preferred embodiment, where it is desired to quantify the transcription level (and thereby expression) of one or more nucleic acid sequences in a sample, the target nucleic acid sample is one in which the concentration of the mRNA transcript(s) of the gene or genes, or the concentration of the nucleic acids derived from the mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear and still provide meaningful results. Thus, for example, an assay where a 5 fold difference in concentration of the target mRNA results in a 3- to 6-fold difference in hybridization intensity is sufficient for most purposes. Where more precise quantification is required, appropriate controls are run to correct for variations introduced in sample preparation and hybridization as described herein. In addition, serial dilutions of “standard” target mRNAs are used to prepare calibration curves according to methods well known to those of skill in the art. Of course, where simple detection of the presence or absence of a transcript is desired, no elaborate control or calibration is required.

For example, if a microarray nucleic acid member is not labeled after hybridization, this indicates that the gene comprising that nucleic acid member is not expressed in either sample. If a nucleic acid member is labeled with a single color, it indicates that a labeled gene was expressed only in one sample. The labeling of a nucleic acid member comprising an array with both colors indicates that the gene was expressed in both samples. Even genes expressed once per cell are detected (1 part in 100,000 sensitivity). A difference in expression intensity in the two samples being compared is indicative of differential expression, the ratio of the intensity in the two samples being not equal to 1.0, preferably less than 0.7 or greater than 1.2, more preferably less than 0.5 or greater than 1.5.

Many human genes are expressed at different levels in cartilage of different developmental (fetal vs. mature) or disease states. In some cases, a gene is not expressed at all in some developmental or disease states, and at high levels in others. Differential analysis of chondrocyte gene expression in differing cartilage states using an EST-based approach is used to identify genes that may play important roles in osteoarthritis pathogenesis and cartilage repair. The advantage of this method is that it can provide gene expression information on a larger scale than other methods. The cDNA clones generated by this approach is useful for future functional studies of certain genes. This type of genomic-based approach can provide important novel insights into our understanding of the osteoarthritis disease process and provide for novel diagnostic, prognostic and therapeutic approaches.

Diagnostic or Prognostic Tests

The invention also provides for diagnostic tests for detecting osteoarthritis. The invention also provides for prognostic tests for monitoring a patient's response to therapy.

According to the method of the invention, mild or severe osteoarthritis is detected by obtaining a cartilage sample from a patient. In alternative embodiments, a blood or synovial fluid sample is obtained from a patient. A sample comprising nucleic acid corresponding to RNA (i.e., RNA or cDNA) is prepared from the patient cartilage (or blood or synovial fluid) sample. The sample comprising nucleic acid corresponding to RNA is hybridized to an array comprising a solid substrate and a plurality of nucleic acid members, where at least one member is differentially expressed in cartilage isolated from a patient diagnosed with mild, moderate, marked or severe osteoarthritis, as compared to a “normal individual”, according to the invention. According to this diagnostic test, hybridization of the sample comprising nucleic acid corresponding to RNA to one or more nucleic acid members on the array is indicative of disease.

A patient response to therapy is monitored by using a prognostic test according to the invention. In one aspect, a prognostic test according to the invention comprises obtaining a cartilage sample from a patient prior to treatment, during the course of treatment and after treatment. Preferably, the patient is treated for at least 12 hours before a sample is taken. In alternative embodiments, blood or synovial fluid samples are obtained from a patient prior to treatment, during the course of treatment and after treatment. A sample comprising nucleic acid corresponding to RNA (i.e., RNA or cDNA) is prepared from the patient cartilage (or blood or synovial fluid) samples. The samples comprising nucleic acid corresponding to RNA are hybridized to an array comprising a solid substrate and a plurality of nucleic acid members, where at least one member is differentially expressed in cartilage isolated from a patient diagnosed with mild, moderate, marked or severe osteoarthritis, as compared to a normal individual, according to the invention. Arrays are selected in accordance with the diagnostic state of the patient whose treatment is being monitored. According to this prognostic test, differential hybridization of the samples comprising nucleic acid corresponding to RNA isolated prior to and after treatment to one or more nucleic acid members on the array is indicative of an effective treatment. Preferably, gene expression profiles in patients being treated changes to resemble more closely gene expression profiles in patients with less severe forms of the disease or more preferably more closely resembles gene expression profiles in normal patients. The extent of change in a gene expression profile can be further correlated with various therapeutic endpoints such as a decrease in the severity and/or occurrence of one or more symptoms associated with the disease.

Confirmation of Differential Expression by Quantitative RT-PCR

Differential expression of biomarkers in samples from individuals with mild or severe osteoarthritis relative to that from normal individuals can also be measured by quantitative RT-PCR (RQ RT-PCR) assay. In one embodiment, any PCR based method able to quantitate mRNA levels is encompanied by the invention. The reverse transcriptase (RT) PCR amplification procedure is a variant of PCR that permits amplification of mRNA templates (either using a one step (combined RT and PCR) or using a two step (RT and subsequent PCR). For quantitative RT-PCR the preferred method of amplifying OA biomarker gene product(s) utilizes fluorogenic hybridization probes or dsDNA-specific fluorescent dyes to detect PCR product during amplification (real-time detection) without purification or separation by gel electrophoresis. The sensitivity of this method's probes allows measurement of the PCR product during the exponential phase of amplification before the critical reactants become limiting.

In one embodiment of the invention, quantitative PCR, more specifically, quantitative RT-PCR, can be used to determine the level of RNA generated from OA biomarkers in a sample. The methods may be semi-quantitative or fully quantitative or may allow relative quantitation. Competitive quantitative PCR® and real-time quantitative PCR®, estimate target gene concentration in a sample by comparison with standard curves constructed from amplifications of serial dilutions of standard DNA. In competitive QPCR, an internal competitor DNA is added at a known concentration to both serially diluted standard samples and unknown (environmental) samples. After coamplification, ratios of the internal competitor and target PCR® products are calculated for both standard dilutions and unknown samples, and a standard curve is constructed that plots competitor-target PCR® product ratios against the initial target DNA concentration of the standard dilutions. Given equal amplification efficiency of competitor and target DNA, the concentration of the latter in environmental samples can be extrapolated from this standard curve.

In real-time QPCR, the accumulation of amplification product is measured continuously in both standard dilutions of target DNA and samples containing unknown amounts of target DNA. A standard curve is constructed by correlating initial template concentration in the standard samples with the number of PCR® cycles (Ct) necessary to produce a specific threshold concentration of product. In the test samples, target PCR® product accumulation is measured after the same C t, which allows interpolation of target DNA concentration from the standard curve.

In another embodiment, one can utilize “relative quantitative PCR,” this method determines the relative concentrations on the specific nucleic acid of interest relative to a control. Each specific nucleic acid is compared with the rate of amplification of the control by measuring the change in Ct (δCt) as between each of the genes of interest as compared with the control. In the context of the present invention, RT is performed on mRNA species isolated from different patients are thus comparable by measuring the δCt for each. Standard internal controls which can be used for this method include 18S, or β-actin. Relative quantitative PCR can be done subsequent to the RT reaction (two-step) or can be done concurrently with the PCR reaction (one-step reaction). By determining that the concentration of a specific mRNA species varies, it is shown that the gene encoding the specific mRNA species is differentially expressed. In one embodiment, at least one of the primers chosen to amplify the sequence of interest is chosen such that the primer spans an intron so that gDNA contamination does not play a crucial role. In another embodiment, the RNA is treated with DNase I (RNase-free).

Thus, the use of such methods to determine the differential expression of osteoarthritic associated nucleic acids and fragments thereof, from among the genes listed in Tables 1-7 with routine methods known to those of ordinary skill in the art, and the expression determined by quantitative or qualitative polynucleotide measurement method thus allowing for the diagnosing of the presence of Osteoarthritis, or a specific stage of osteoarthritis, such as severe or, or a prognostic method for selecting treatment strategies for osteoarthritis patients.

### LITERATURE

Celi F S, Zenilman M E, Shuldiner A R. 1993: A rapid and versatile method to synthesize internal standards for competitive PCR. Nucleic Acids Research 21; p. 1047
Schneeberger C, Speiser P, Kury F, Zeillinger R. 1995: Quantitative detection of reverse transcriptase-PCR products by means of a novel and sensitive DNA stain. PCR Methods and Applications 4; p. 234-238
Quantitative RT-PCR. Methods and Applications Book 3; Clontech Laboratories, Inc.
Jack Vanden Heuvel, PCR Protocols in Molecular Toxicology in Methods in Life Sciences—Toxicology Section Volume: 4, CRC Press (1997)
Rychlik, W. and Rhoads, R. E. (1989) A computer program for choosing optimal oligonucleotides for filter hybridization, sequencing and in vitro amplification of DNA. Nucl. Acids Res. 17: 8543-8551.
Gilliland, G. S., Perrin, K. and Bunn, H. F. Competitive PCR for quantitation of mRNA. In: Innis et al. (eds.) PCR Protocols: A Guide to Methods and Applications, pp. 60-66, San Diego, Calif., Academic Press, Inc., 1990.
Vanden Heuvel, J. P., Tyson, F. L. and Bell, D. A. (1993) Constructions of recombinant RNA templates for use as internal standards in quantitative RT-PCR. Biotechniques 14: 395-398.
Vanden Heuvel, J. P., Clark, G. C., Kohn, M. C., Tritscher, A. M., Greenlee, W. F., Lucier, G. W. and Bell, D. A. (1994) Dioxin-responsive genes: Examination of dose-response relationships using quantitative reverse transcriptase-polymerase chain reaction. Cancer Res. 54: 62-68.

### EXAMPLES

The examples below are non-limiting and are merely representative of various aspects and features of the present invention.

### Example 1

### RNA Extraction and Normal Adult cDNA Library Construction

A cDNA library was prepared from normal adult cartilage. ESTs were obtained from the cDNA library and characterized to create one or more gene expression profiles for normal adult chondrocytes.

Large-Scale Sequencing of cDNA Inserts

cDNA libraries were constructed into λTripleEx2 vector through a PCR-based method, using SMART (Switching Mechanism At 5′ end of RNA Transcript) cDNA Library Construction Kit (Clontech). Phage plaques were randomly picked and positive inserts were identified by PCR. Agarose gel electrophoresis was used to assess the presence and purity of inserts. PCR product was then subjected to automated DNA sequencing with a 5′ vector-specific forward primer and sequenced by ABI PRISM 377 DNA sequencer (Perkin Elmer) and ABI PRISM 3700 DNA Analyzer (Applied Biosystems). All generated EST sequences were searched against the nonredundant Genebank/EMBL/DDBL, dbEST and GSS databases. A minimum value of p=10⁻¹⁰ and nucleotide sequence identity >90% were required for assignments of putative identities for EST-matching to known genes or other ESTs. Relative EST frequency level was calculated by dividing the EST number matched to that gene into the total number of ESTs obtained from the library.

Sequences were manually edited or edited using Sequencher software (GeneCodes). All edited EST sequences were compared to the non-redundant Genbank/EMBL/DDBJ and dbEST databases using the BLAST algorithm (8). A minimum value of P=10⁻¹⁰ and nucleotide sequence identity >95% were required for assignments of putative identities for ESTs matching to known genes or to other ESTs. Construction of a non-redundant list of genes represented in the EST set was done with the help of Unigene, Entrez and PubMed at the National Center for Biotechnology Information (NCBI) site (http://www.ncbi.nlm.nih.gov/). Relative gene expression frequency was calculated by dividing the number of EST copies for each gene by the total number of ESTs analyzed. Functional characterization of ESTs with known gene matches was made according to the categories described by Hwang et al (Hwang D M, Dempsey A A, Wang R X, Rezvani M, Barrans J D, Dai K S, et al. A Genome-Based Resource for Molecular Cardiovascular Medicine: Toward a Compendium of Cardiovascular Genes. Circulation 1997; 96:4146-203).

### Example 2

### RNA Extraction and cDNA Library Construction from Mild Osteoarthritic Chondrocytes and Severe Osteoarthritic Chondrocytes

A cDNA library was prepared from mild osteoarthritic cartilage and severe osteoarthritic cartilage. ESTs were obtained from the cDNA libraries and characterized to create one or more gene expression profiles for mild osteoarthritic chondrocytes and severe osteoarthritic chondrocytes.

Articular cartilage was obtained during either arthroscopic knee surgery or total knee replacement. The cartilage samples were obtained from either areas of very early cartilage degeneration (mild) or from sites of end stage disease (severe). cDNA libraries were constructed as described for normal adult samples (Example 1).

Large-Scale Sequencing of cDNA Inserts

cDNA libraries were constructed into λTripleEx2 vector through a PCR-based method, using SMART (Switching Mechanism At 5′ end of RNA Transcript) cDNA Library Construction Kit (Clontech). Phage plaques were randomly picked and positive inserts were identified by PCR. Agarose gel electrophoresis was used to assess the presence and purity of inserts. PCR product was then subjected to automated DNA sequencing with a 5′ vector-specific forward primer and sequenced by ABI PRISM 377 DNA sequencer (Perkin Elmer) and ABI PRISM 3700 DNA Analyzer (Applied Biosystems). All generated EST sequences were searched against the nonredundant Genebank/EMBL/DDBL, dbEST and GSS databases. A minimum value of p=10⁻¹⁰ and nucleotide sequence identity >90% were required for assignments of putative identities for EST-matching to known genes or other ESTs. Relative EST frequency level was calculated by dividing the EST number matched to that gene into the total number of ESTs obtained from the library.

Sequences were manually edited or edited using Sequencher software (GeneCodes). All edited EST sequences were compared to the non-redundant Genbank/EMBL/DDBJ and dbEST databases using the BLAST algorithm (8). A minimum value of P=10⁻¹⁰ and nucleotide sequence identity >95% were required for assignments of putative identities for ESTs matching to known genes or to other ESTs.

Construction of a non-redundant list of genes represented in the EST set was done with the help of Unigene, Entrez and PubMed at the National Center for Biotechnology Information (NCBI) site (http://www.ncbi.nlm.nih.gov/). Relative gene expression frequency was calculated by dividing the number of EST copies for each gene by the total number of ESTs analyzed.

Functional characterization of ESTs with known gene matches was made according to the categories described by Hwang et al (Hwang D M, Dempsey A A, Wang R X, Rezvani M, Barrans J D, Dai K S, et al. A Genome-Based Resource for Molecular Cardiovascular Medicine: Toward a Compendium of Cardiovascular Genes. Circulation 1997; 96:4146-203).

### Example 3

### Microarray Construction

A microarray according to the invention was constructed as follows.

PCR products (˜40 μl) of cDNA clones from OA cartilage cDNA libraries, in the same 96-well tubes used for amplification, are precipitated with 4 μl ( 1/10 volume) of 3M sodium acetate (pH 5.2) and 100 μl (2.5 volumes) of ethanol and stored overnight at −20° C. They are then centrifuged at 3,300 rpm at 4° C. for 1 hour. The obtained pellets were washed with 50 μl ice-cold 70% ethanol and centrifuged again for 30 minutes. The pellets are then air-dried and resuspended well in 50% dimethylsulfoxide (DMSO) or 20 ul 3×SSC overnight. The samples are then deposited either singly or in duplicate onto Gamma Amino Propyl Silane (Corning CMT-GAPS or CMT-GAP2, Catalog No. 40003, 40004) or polylysine-coated slides (Sigma Cat. No. P0425) using a robotic GMS 417 or 427 arrayer (Affymetrix, CA). The boundaries of the DNA spots on the microarray are marked with a diamond scriber. The invention provides for arrays where 10-20,000 PCR products are spotted onto a solid support to prepare an array.

The arrays are rehydrated by suspending the slides over a dish of warm particle free ddH₂O for approximately one minute (the spots will swell slightly but not run into each other) and snap-dried on a 70-80° C. inverted heating block for 3 seconds. DNA is then UV crosslinked to the slide (Stratagene, Stratalinker, 65 mJ—set display to “650” which is 650×100 μJ) or baked at 80 C for two to four hours. The arrays are placed in a slide rack. An empty slide chamber is prepared and filled with the following solution: 3.0 grams of succinic anhydride (Aldrich) is dissolved in 189 ml of 1-methyl-2-pyrrolidinone (rapid addition of reagent is crucial); immediately after the last flake of succinic anhydride dissolved, 21.0 ml of 0.2 M sodium borate is mixed in and the solution is poured into the slide chamber. The slide rack is plunged rapidly and evenly in the slide chamber and vigorously shaken up and down for a few seconds, making sure the slides never leave the solution, and then mixed on an orbital shaker for 15-20 minutes. The slide rack is then gently plunged in 95° C. ddH₂O for 2 minutes, followed by plunging five times in 95% ethanol. The slides are then air dried by allowing excess ethanol to drip onto paper towels. The arrays are then stored in the slide box at room temperature until use.

### Example 4

### Target Nucleic acid Preparation and Hybridization

Preparation of Fluorescent DNA Probe from mRNA

Fluorescently labeled target nucleic acid samples are prepared for analysis with an array of the invention.

2 μg Oligo-dT primers are annealed to 2 μg of mRNA isolated from a cartilage sample from patient diagnosed with osteoarthritis or suspected of having osteoarthritis in a total volume of 15 μl, by heating to 70° C. for 10 min, and cooled on ice. The mRNA is reverse transcribed by incubating the sample at 42° C. for 1.5-2 hours in a 100 μl volume containing a final concentration of 50 mM Tris-HCl (pH 8.3), 75 mM KCl, 3 mM MgCl2, 25 mM DTT, 25 mM unlabeled dNTPs, 400 units of Superscript II (200 U/μL, Gibco BRL), and 15 mM of Cy3 or Cy5 (Amersham). RNA is then degraded by addition of 15 μl of 0.1N NaOH, and incubation at 70 C for 10 min. The reaction mixture is neutralized by addition of 15 μl of 0.1N HCl, and the volume is brought to 500 μl with TE (10 mM Tris, 1 mM EDTA), and 20 μg of CotI human DNA (Gibco-BRL) is added.

The labeled target nucleic acid sample is purified by centrifugation in a Centricon-30 micro-concentrator (Amicon). If two different target nucleic acid samples (e.g., two samples derived from different patients) are being analyzed and compared by hybridization to the same array, each target nucleic acid sample is labeled with a different fluorescent label (e.g., Cy3 and Cy5) and separately concentrated. The separately concentrated target nucleic acid samples (Cy3 and Cy5 labeled) are combined into a fresh centricon, washed with 500 μlTE, and concentrated again to a volume of less than 7 μl. 1 μL of 10 μg/μl polyA RNA (Sigma, #P9403) and 1 of 10 μg/μl tRNA (Gibco-BRL, #15401-011) is added and the volume is adjusted to 9.5 μl with distilled water. For final target nucleic acid preparation 2.1 μl 20×SSC (1.5M NaCl, 150 mM NaCitrate (pH8.0)) and 0.35 μl 10% SDS is added.

Hybridization

Labeled nucleic acid is denatured by heating for 2 min at 100° C., and incubated at 37° C. for 20-30 min before being placed on a nucleic acid array under a 22 mm×22 mm glass cover slip. Hybridization is carried out at 65° C. for 14 to 18 hours in a custom slide chamber with humidity maintained by a small reservoir of 3×SSC. The array is washed by submersion and agitation for 2-5 min in 2×SSC with 0.1% SDS, followed by 1×SSC, and 0.1×SSC. Finally, the array is dried by centrifugation for 2 min in a slide rack in a Beckman GS-6 tabletop centrifuge in Microplus carriers at 650 RPM for 2 min.

### Example 5

### Signal Detection and Data Generation

Following hybridization of an array with one or more labeled target nucleic acid samples, arrays are scanned immediately using a GMS Scanner 418 and Scanalyzer software (Michael Eisen, Stanford University), followed by GeneSpring software (Silicon Genetics, CA) analysis. Alternatively, a GMS Scanner 428 and Jaguar software may be used followed by GeneSpring software analysis.

If one target nucleic acid sample is analyzed, the sample is labeled with one fluorescent dye (e.g., Cy3 or Cy5).

After hybridization to a microarray as described in Example 4, fluorescence intensities at the associated nucleic acid members on the microarray are determined from images taken with a custom confocal microscope equipped with laser excitation sources and interference filters appropriate for the Cy3 or Cy5 fluors.

The presence of Cy3 or Cy5 fluorescent dye on the microarray indicates hybridization of a target nucleic acid and a specific nucleic acid member on the microarray. The intensity of Cy3 or Cy5 fluorescence represents the amount of target nucleic acid which is hybridized to the nucleic acid member on the microarray, and is indicative of the expression level of the specific nucleic acid member sequence in the target sample.

When two target nucleic acid samples are being analyzed and compared (e.g., mild osteoarthritic vs severe osteoarthritic), one target nucleic acid sample (for example, mild osteoarthritic) is labeled with fluorescent dye Cy3, the other target nucleic acid sample (for example, severe osteoarthritis) is labeled with fluorescent dye Cy5.

After hybridization as described in Example 4, fluorescence intensities at the associated nucleic acid members on the microarray are determined from images taken with a custom confocal microscope equipped with laser excitation sources and interference filters appropriate for the Cy3 and Cy5 fluors. Separate scans are taken for each fluor at a resolution of 225 μm² per pixel and 65,536 gray levels. Normalization between the images is used to adjust for the different efficiencies in labeling and detection with the two different fluors. This is achieved by manual matching of the detection sensitivities to bring a set of internal control genes to nearly equal intensity followed by computational calculation of the residual scalar required for optimal intensity matching for this set of genes.

The presence of Cy3 or Cy5 fluorescent dye on the microarray indicates hybridization of a target nucleic acid and a specific nucleic acid member on the microarray. The intensities of Cy3 or Cy5 fluorescence represent the amount of target nucleic acid which is hybridized to the nucleic acid member on the microarray, and is indicative of the expression level of the specific nucleic acid member sequence in the target sample. If a nucleic acid member on the array shows no color, it indicates that the gene in that element is not expressed in either sample. If a nucleic acid member on the array shows a single color, it indicates that a labeled gene is expressed only in that cell sample. The appearance of both colors indicates that the gene is expressed in both tissue samples. The ratios of Cy3 and Cy5 fluorescence intensities, after normalization, are indicative of differences of expression levels of the associated nucleic acid member sequence in the two samples for comparison. A ratio of expression intensity not equal to 1.0 is used as an indication of differential gene expression.

The array is scanned in the Cy 3 and Cy5 channels and stored as separate 16-bit TIFF images. The images are incorporated and analysed using Scanalyzer software which includes a gridding process to capture the hybridization intensity data from each spot on the array. The fluorescence intensity and background-subtracted hybridization intensity of each spot is collected and a ratio of measured mean intensities of Cy5 to Cy3 is calculated. A liner regression approach is used for normalization and assumes that a scatter plot of the measured Cy5 versus Cy3 intensities should have a scope of one. The average of the ratios is calculated and used to rescale the data and adjusts the slope to one. A post-normalization cutoff of a ratio not equal to 1.0 is used to identify differentially expressed genes.

### Example 6

### Chondrocyte-Specific Gene Microarray And Diagnosis Microarray Construction

A collection of nucleic acid members can be spotted on a glass slide as described in Example 3 for the construction of either an OA diagnostic microarray, a mild OA diagnostic microarray or a severe OA diagnosis microarray or a chondrocyte specific gene microarray. The nucleic acid members spotted onto the microarrays described are selected from those named in Tables 1-7.

### Example 7

### Diagnosis

Microarray Analysis

Target nucleic acid samples are prepared from cartilage RNA extracts of an individual (as described in Example 2 and hybridized to a microarray comprising a collection of nucleic acid members where at least one member is differentially expressed in cartilage isolated from a patient diagnosed with mild or severe osteoarthritis, as compared to cartilage isolated from a normal individual as defined herein (as described in Example 1). A hybridization pattern is generated and analyzed as in Example 5. For example, the hybridization of target nucleic acid samples to one or more nucleic acid members on the microarray comprising a collection of nucleic acid members where at least one member is differentially expressed in mild osteoarthritis cartilage as compared to a normal individual is indicative of a mild osteoarthritis in the individual from whom the target nucleic acid sample is derived. The hybridization of target nucleic acid samples to one or more nucleic acid members on the microarray comprising a collection of nucleic acid members differentially expressed in severe osteoarthritis cartilage as compared to the normal individual is indicative of severe osteoarthritis in the individual from whom the target nucleic acid sample is derived.

RT-PCR Analysis

Diagnosis of OA with Cartilage Samples

Diagnostic and prognostic methods that utilize gene expression data from one or more OA biomarkers through the use of expression level ratios and rationally chosen thresholds have been developed. The effectiveness of OA biomarkers in diagnosing OA and stages of OA is demonstrated. Biomarkers thus identified can be measured using any technique known to measure gene expression of RNA. For example one can utilize real time quantitative reverse-transcriptase polymerase chain reaction. Real time quantitative reverse-transcriptase assays can easily adapted to a clinical setting to diagnose OA, or a specific stage of OA. Accordingly, diagnostic assays for classification of OA, selecting and monitoring treatment regimens, and monitoring OA progression/regression can be done utilizing QRT-PCR. This type of assay is particularly suited to monitoring smaller numbers of OA Biomarker genes.

In one embodiment, the method utilized encompasses (a) obtaining a cartilage sample from an individual to be diagnosed; (b) obtaining RNA transcripts from said sample; (c) performing quantitative PCR® on said RNA using primers that amplify a nucleic acid segment selected from the group of biomarker genes listed in Table 1 in conjunction with Sybr® green; and (d) comparing the amount of amplification product of said biomarker genes as compared with a control (δCt) (e) comparing the (δCt) in (d) with the (δCt) of the amount of amplification product in cartilage from an individual not having osteoarthritis as compared with the same control, wherein an increase or a decrease in the relative amount of OA biomarker amplification product in samples of said individual, as compared to the amount of the same OA Biomarker amplification product in an equivalent sample from a normal individual, indicates that said subject has OA.

### Example 8

### Assessing the Integrity of Cartilage RNA Isolated Post-Mortem

The following Baboon cartilage study was performed to evaluate the quality of freshly isolated RNA and RNA isolated at various times post-mortem.

Nine vials of baboon cartilage were obtained, and stored in liquid nitrogen till use.

Baboon cartilage from each vial was weighed and finely powdered under liquid nitrogen. The sample was then homogenised in TRIzol® reagent (0.1 g/ml TRIzol®) and total RNA was extracted. The quantity of RNA was calculated according to the OD₂₆₀ value. The appearance of two sharp bands on the RNA gel indicated that the RNA was of good quality.

RT-PCR was performed for the gene expression of collagen type II (COL2A1), B-actin and GAPDH, using 0.1 ug total RNA from each sample.

The RNA gel pattern clearly shows that the RNA was not degraded up to 12 hours post mortem (Table 10). Therefore stable RNA should be expected from the biopsy sample within 12 hours after death.

TABLE 10
Integrity Of Cartilage RNA Isolated Post-Mortem

| | | | Total RNA | | | | |
|---|---|---|---|---|---|---|---|
| Sample | Time | | (ug) - Based | RNA Gel | | | |
| No. | Taken | Weight (g) | on OD260 | (non Dil) | Col2A1 | actin | GAPDH |
| 1 | Fresh | 0.175 | 8 | OK | ++ | ++ | ++ |
| 2 | 1 hr pm | 0.29 | 9 | OK | ++ | ++ | ++ |
| 3 | 2 hr | 0.29 | 11.36 | OK | ++ | +/− | +/− |
| 4 | 3 hr | 0.25 | 2.8 | OK | ++ | +/− | +/− |
| 5 | 6 hr | 0.53 | 8.0 | OK | ++ | + | +/− |
| 6 | 8 hr | 0.18 | 5.26 | OK | ++ | + | − |
| 7 | 10 hr | 0.38 | 9.35 | OK | ++ | + | +/− |
| 8 | 12 hr | 0.20 | 6.7 | OK | ++ | +/− | − |
| 9 | 24 hr | 0.41 | 9.35 | SMEAR | +/− | − | − |

Collagen type II is abundant and specific to normal articular cartilage. Its mRNA level was comparable among all the samples except #9 (24 hours post-mortem). It should be noted that samples taken earlier will better reflect the natural in vivo state.

### Example 9

### Expressed Sequence Tags (ESTs) Analysis of Human Chondrocyte Gene Expression in Mild and Severe Osteoarthritic Cartilage

Large scale sequencing of cDNA libraries from human normal, mild and severe OA cartilage was also performed and several thousands of ESTs from the three cDNA libraries were analysed.

Normal cartilage was obtained from the donor program of Department of Orthopedics and Rehabilitation, University of Miami. OA cartilage samples were obtained from either areas of very early cartilage degeneration (mild) or from sites of end stage disease (severe) during either arthroscopic knee surgery or total knee replacement. Total RNA from cartilage was extracted using TRIzol® reagent (GIBCO). cDNA libraries were constructed into λTriplEx2 vector through a PCR-based method, using SMART (Switching Mechanism At 5′ end of RNA Transcript) cDNA Library Construction Kit (Clontech) as described above. Phage plaques were randomly picked and positive inserts were identified by PCR. Agarose gel electrophoresis was used to assess the presence and purity of inserts. PCR product was then subjected to automated DNA sequencing with a 5′ vector-specific forward primer and sequenced by ABI PRISM 377 DNA sequencer (Perkin Elmer) and ABI PRISM 3700 DNA Analyzer (Applied Biosystems). All generated EST sequences were searched against the nonredundant Genebank/EMBL/DDBL, dbEST and GSS databases. A minimum value of p=10⁻¹⁰ and nucleotide sequence identity >90% were required for assignments of putative identities for EST-matching to known genes or other ESTs. Relative EST frequency level was calculated by dividing the EST number matched to that gene into the total number of ESTs obtained from the library.

Several thousand ESIs were obtained from normal, mild and severe OA cDNA libraries respectively and used for gene expression profiling. Differentially expressed known genes amongst normal, mild, and severe OA cartilage were identified by examining relative EST frequency levels and are shown in Tables 1-7.

### Example 10

### Identification of OA Biomarkers

One effective and rapid way of characterizing gene expression patterns in chondrocytes from individuals with OA relative to that in normal chondrocytes and thus identifying OA biomarkers is by using an EST analysis. An EST analysis refers to the relative expression level of a gene based on the frequency of ESTs representing the gene derived from an OA chondrocyte cDNA library as compared to the frequency of ESTs representing the same gene derived from a normal chondrocyte cDNA library. The relative EST frequency of an EST is calculated by dividing the number of ESTs representing each specific gene by the total number of ESTs analyzed. Differences in relative EST frequency may be used as an indication of differential gene expression between normal chondrocytes and OA chondrocytes since cDNA libraries represent gene transcription in the chondrocytes used to construct the library. Table 1 shows sequences identified in the normal, mild osteoarthritic and severe osteoarthritic cDNA libraries and the relative EST frequency of each gene in each library. One of skill can easily envision identifying an OA biomarker gene from Table 1, based on the biomarker gene's differential expression in OA chondrocytes relative to normal chondrocytes.

### Example 11

### Identification of Specific Mild OA Biomarkers

A matrix to identify mild OA specific biomarkers is illustrated in FIGS. 1, 3 and 5, and encompasses comparison of a gene displaying an increase number of ESTs in the mild OA cDNA library relative to the cDNA library from normal chondrocytes, with the frequency of the comparison of the EST frequency in the severe OA cDNA library relative to the cDNA library from normal chondrocytes for the same gene. FIG. 3 uses more stringent selection criteria regarding which genes will be compared based on each gene's respective EST frequency, while FIG. 5 uses the most stringent selection criteria. The EST frequency of each of these 5,687 genes in each of these three different libraries is also listed in Table 1.

These listed EST frequencies have been analyzed using a matrix to identify biomarkers specific for mild OA. Three matrices with different stringencies with respect to the criteria used to identify biomarkers were developed. The first matrix compares the regulation of a gene (up regulated, down regulated, and no regulation as determined by the relative frequency of EST) in chondrocytes from individuals with mild OA with respect to chondrocytes from normal individual(s). A gene is determined to be differentially expressed in chondrocytes from individuals with mild OA according to this first matrix, as long as the gene does not display the same direction of change (i.e. up or down) in EST frequency as do chondrocytes from individuals with severe OA, (FIG. 1). Table 2 lists the mild OA stage specific gene biomarkers generated using the first matrix.

The second matrix is similar in principle to the first matrix, however the genes used for comparison in the second matrix are selected from those genes wherein the designated regulation (up regulated, down regulated, and no regulation) is determined with reference to the p value being less than 0.05 as indicated by the relative frequency of EST in chondrocytes from individuals with mild OA with respect to chondrocytes from normal individual(s). As described above, a gene is determined to be differentially expressed in chondrocytes from individuals with mild OA according to this first matrix, as long as the gene does not display the same direction of change (i.e. up or down) in EST frequency as do chondrocytes from individuals with severe OA, (FIG. 3). 35 such genes were identified as being biomarkers of mild OA, (Table 4).

The third matrix which is based on the second matrix, generates yet another subset of OA stage specific biomarkers by further requiring that a mild OA stage specific biomarker which is upregulated in mild OA chondrocytes with a p value being less than 0.05 relative to its expression in normal chondrocytes, must also be down regulated with a p value being less than 0.05 in chondrocytes with severe OA relative to normal chondrocytes. Conversely, a mild OA stage specific OA biomarker which is downregulated in mild OA chondrocytes with statistical significance relative to its expression in normal chondrocytes, must also be up regulated with statistical significance in chondrocytes with severe OA relative to normal chondrocytes. Table 6 lists 9 mild OA stage specific biomarker genes.

### Example 12

### Identification of Specific Severe OA Biomarkers

Similarly, a matrix to identify severe OA specific biomarkers is illustrated in FIGS. 2, 4 and 6, and encompasses comparison of a gene displaying an increase number of ESTs in the severe OA cDNA library relative to the cDNA library from normal chondrocytes, with the frequency of the comparison of the EST frequency in the mild OA cDNA library relative to the cDNA library from normal chondrocytes for the same gene.

The first matrix compares the regulation of a gene (up regulated, down regulated, and no regulation as determined by the relative frequency of EST) in chondrocytes from individuals with severe OA with respect to chondrocytes from normal individual(s). A gene is determined to be differentially expressed in chondrocytes from individuals with severe OA according to this first matrix, as long as the gene does not display the same direction of change (i.e. up or down) in EST frequency as do chondrocytes from individuals with mild OA, (FIG. 2).

The second matrix as described above, is similar in principle to the first matrix, however the genes used for comparison in the second matrix are selected from those genes wherein the designated regulation (up regulated, down regulated, and no regulation) is determined to be statistically significant as indicated by the relative frequency of EST in chondrocytes from individuals with severe OA with respect to chondrocytes from normal individual(s). As described above, a gene is determined to be differentially expressed in chondrocytes from individuals with severe OA according to this matrix, as long as the gene does not display the same direction of change (i.e. up or down) in EST frequency as do chondrocytes from individuals with mild OA, (FIG. 4). 49 such genes were identified as being biomarkers of severe OA, (Table 5).

The third matrix which is based on the second matrix, generates yet another subset of OA stage specific biomarkers by further requiring that a severe OA stage specific biomarker which is upregulated in severe OA chondrocytes with a p value being less than 0.05 relative to its expression in normal chondrocytes, must also be down regulated with statistical significance in chondrocytes with mild OA relative to normal chondrocytes. Conversely, a severe OA stage specific OA biomarker which is downregulated in severe OA chondrocytes with a p value being less than 0.05 relative to its expression in normal chondrocytes, must also be up regulated with statistical significance in chondrocytes with mild OA relative to normal chondrocytes. Table 7 lists 10 severe OA stage specific biomarker genes.

Variations, modifications, and other implementations of what is described herein will occur to those of ordinary skill in the art without departing from the spirit and scope of the invention. The references provided below are incorporated herein by reference in their entireties. All patents, patent applications, and published references cited herein are hereby incorporated by reference in their entirety.

While this invention has been particularly shown and described with references to preferred embodiments thereof, it will be understood by those skilled in the art that various changes in form and details may be made therein without departing from the scope of the invention encompassed by the appended claims. Those skilled in the art will recognise that other embodiments and configurations known in the art would be within the spirit and scope of the present invention.

### REFERENCES

1. Zaleske D J. Cartilage and Bone Development. Instr Course Lect 1998; 47:461-
2. Buckwalter J A, Mankin H J. Articular Cartilage: Tissue Design and Chondrocyte-Matrix Interactions. Instr Course Lect 1998; 47:477-86.
3. Westacott C I, Sharif M. Cytokines in Osteoarthritis: Mediators or Markers of Joint Destruction? Semin Arthritis Rheum 1996; 25:254-72
4. Adams M D, Kerlavage A R, Fleischmann R D, Fuldner R A, Bult C J, Lee N H, et al. Initial assessment of human gene diversity and expression patterns based upon 83 million nucleotides of cDNA sequence. Nature 1995; 377 Suppl:3-174.
5. Hwang D M, Dempsey A A, Wang R X, Rezvani M, Barrans J D, Dai K S, et al. A Genome-Based Resource for Molecular Cardiovascular Medicine: Toward a Compendium of Cardiovascular Genes. Circulation 1997; 96:4146-203.
6. Mao M, Fu G, Wu J S, Zhang Q H, Zhou J, Kan L X, et al. Identification of genes expressed in human CD34⁺ hematopoietic stem/progenitor cells by expressed sequence tags and efficient full-length cDNA cloning. Proc Natl Acad Sci 1998; 95:8175-80.
7. Hillier L D, Lennon G, Becker M, Bonaldo M F, Chiapelli B, Chissoe S, et al. Generation and analysis of 280,000 human expressed sequence tags. Genome Res. 1996; 6:807-28.
8. Altschul S F, Gish W, Miller W, Myers E W, Lipman D J. Basic local alignment search tool. J Mol Biol 1990; 215:403-10.
9. Mundlos S, Zabel B. Developmental Expression of Human Cartilage Matrix Protein. Dev Dyn 1994; 199:241-52.
10. Nakamura S, Kamihagi K, Satakeda H, Katayama M, Pan H, Okamoto H, et al. Enhancement of SPARC (osteonectin) synthesis in arthritic cartilage. Increased levels in synovial fluids from patients with rheumatoid arthritis and regulation by growth factors and cytokines in chondrocyte cultures. Arthritis Rheum 1996; 39:539-51.
11. Eyre D R, The Collagens of Articular Cartilage. Semin Arthritis Rheum 1991; 21 (3 Suppl 2):2-11.
12. Okihana H, Yamada K. Preparation of a cDNA Library and Preliminary Assessment of 1400 Genes from Mouse Growth Cartilage. J Bone Miner Res 1999; 14:304-10.
13. Morrison E H, Ferguson M W J, Bayliss M T, Archer C W. The developmental of articular cartilage: I. The spatial and temporal patterns of collagen types. J Anat 1996; 189:9-22.
14. Treilleux I, Mallein-Gerin F, le Guellec D, Herbage D. Localization of the Expression of Type I, II, III Collagens, and Aggrecan Core Protein Genes in Developing Human Articular Cartilage. Matrix 1992; 12:221-32.
15. Eyre D R, Wu J J, Niyibizi C. The collagens of bone and cartilage: Molecular diversity and supramolecular assembly. In Cohn D V, Glorieux F H, Martin T J, editors. Calcium Regulation and Bone Metabolism. Amsterdam. The Netherlands: Elsevier; 1990. p. 188-94.
16. Bimbacher R. Amann G, Breitschopf H, Lassmann H, Suchanek G, Heinz-Erian P. Cellular localization of insulin-like growth factor II mRNA in the human fetus and the placenta: detection with a digoxigenin-labeled cRNA probe and immunocytochemistry. Pediatr Res 1998; 43:614-20.
17. Wang E, Wang J, Chin E, Zhou J, Bondy C A. Cellular patterns of insulin-like growth factor system gene expression in murine chondrogenesis and osteogenesis. Endocrinology 1995; 136:2741-51.
18. van Kleffens M, Groffen C, Rosato R R, van den Eijnde S M, van Neck J W, Lindenbergh-Kortleve D J, et al. mRNA expression patterns of the IGF system during mouse limb bud development, determined by whole mount in situ hybridization. Mol Cell Endocrinol 1998; 138:151-61.
19. Braulke T, Gotz W, Claussen M. Immunohistochemical localization of insulin-like growth factor binding protein-1, -3, and -4 in human fetal tissues and their analysis in media from fetal tissue explants. Growth Regul 1996; 6:55-65.
20. Kessler E, Takahara K, Biniaminov L, Brusel M, Greenspan D S. Bone Morphogenetic Protein-1: The Type I Procollagen C-Proteinase. Science 1996; 271:360-2.
21. Ausubel et al., John Weley & Sons, Inc., 1997, Current Protocols in Molecular Biology
22. Marshall, K. et al., 2000, 46^{th} Annual Meeting, ORS, paper No. 919.
23. Kumar, S., et al., 2000, 46^{th} Annual Meeting, ORS, paper No. 1031.
24. Marshall K., et al., 2002, 48^{th} Annual meeting, ORS (submitted).
25. Migita K., et al., Biochem Biophys Res Commun 1997, 239:621-625.
26. Migita K., et al., Kidney Int 1999, 55:572-578.
28. Stephane Audic and J-M Clayerie 1997, Genome Research Vol 7. No. 10 p. 986-995.

1. A method of diagnosing Osteoarthritis (OA) in an individual, comprising determining the level of one or more RNA transcripts which correspond to one or more genes respectively, selected from Table 1 in a sample from an individual suspected of having or being afflicted with OA, wherein a difference in the level of said one or more RNA transcripts in said individual compared to the level of said one or more RNA transcripts in a normal individual is indicative of OA.

2. A method of diagnosing mild Osteoarthritis (OA) in an individual, comprising determining the level of one or more RNA transcripts corresponding to one or more genes respectively, selected from Table 1 in a sample from an individual suspected of having or being afflicted with mild OA, wherein a difference in expression of said one or more RNA transcripts in said individual compared to the expression of said one or more RNA transcripts in a normal individual is indicative of mild OA.

3. The method of claim 2, wherein said one or more genes is selected from any one of the genes listed in Table 2.

4. The method of claim 2, wherein said one or more genes is selected from any one of the genes listed in Table 4.

5. The method of claim 2, wherein said one or more genes is selected from any one of the genes listed in Table 6.

6. A method of diagnosing severe Osteoarthritis (OA) in an individual, comprising determining the level of one or more RNA transcripts corresponding to one or more genes respectively, selected from Table 1 in a sample from an individual suspected of having or being afflicted with severe OA, wherein a difference in expression of said one or more RNA transcripts in said individual compared to the expression of said one or more RNA transcripts in a normal individual is indicative of severe OA.

7. The method of claim 6, wherein said one or more genes is selected from any one of the genes listed in Table 3.

8. The method of claim 6, wherein said one or more genes is selected from any one of the genes listed in Table 5.

9. The method of claim 6, wherein said one or more genes is selected from any one of the genes listed in Table 7.

10. The method of any one of claims 1-9, further comprising the step of isolating RNA from said patient.

11. The method of claim 10, further comprising the step of isolating RNA from a blood sample.

12. The method of claim 10, further comprising the step of isolating RNA from a synovial fluid sample.

13. The method of claim 10, further comprising the step of isolating RNA from a cartilage sample.

14. The method of claim 10, wherein said cartilage sample comprises cartilage isolated from cartilage tissue less than 14 hours post-mortem.

15. The method of any one of claims 1-9, wherein said determining the level of said RNA transcripts comprises hybridizing a nucleic acid sample comprising or corresponding to said RNA transcripts corresponding to genes selected from Table 1 from an individual suspected of having or being afflicted with OA, to an array comprising a solid substrate and a plurality of nucleic acid members, wherein at least two of said nucleic acid members are differentially expressed in cartilage isolated from a patient diagnosed with Osteoarthritis as compared to cartilage isolated from a normal individual, wherein each nucleic acid member has a unique position and is stably associated with the solid substrate, and wherein hybridization of said nucleic acid sample to said differentially expressed nucleic acid members is indicative of Osteoarthritis.


SEQUENCE LISTING (2 sequences)

SEQ ID NO: 1 - DNA, 31 bp, Artificial
  modified T3 phage primer
gccaagctcgaaattaaccctcactaaaggg

SEQ ID NO: 2 - DNA, 33 bp, Artificial
  modified T7 phage primer
ccagtgaattgtaatacgactcactatagggcg